SEQUENCE LISTING (49 sequences)

SEQ ID NO: 1 - protein, 12 aa, synthetic construct
EAKIEKHNHYQK

SEQ ID NO: 2 - protein, 25 aa, synthetic construct
EAKIEKHNHYQKGEAKIEKHNHYQK

SEQ ID NO: 3 - protein, 24 aa, synthetic construct
MGIPMGKSMLVLLTFLAFASCCIA

SEQ ID NO: 4 - protein, 19 aa, synthetic construct
TFFYGGSRGKRNNFKTEEY

SEQ ID NO: 5 - protein, 39 aa, synthetic construct
SSVIDALQYKLEGTTRLTRKRGLKLATALSLSNKFVEGS

SEQ ID NO: 6 - protein, 18 aa, synthetic construct
LRKLRKRLLLRKLRKRLL

SEQ ID NO: 7 - protein, 8 aa, synthetic construct
  SITE 8: note = Amidated
  SITE 1: note = Acetylated
  REGION 1..8: note = Circular
CMPRLRGC

SEQ ID NO: 8 - protein, 12 aa, synthetic construct
  SITE 12: note = C- terminal amidation
THRPPMWSPVWP

SEQ ID NO: 9 - protein, 12 aa, synthetic construct
  SITE 12: note = C- terminal amidation
  SITE 1: note = D-proline
  SITE 2: note = D-tryptophan
  SITE 3: note = D-valine
  SITE 4: note = D-proline
  SITE 5: note = D-serine
  SITE 6: note = D-tryptophan
  SITE 7: note = D-methionine
  SITE 8: note = D-proline
  SITE 9: note = D-proline
  SITE 10: note = D-arginine
  SITE 11: note = D-histidine
  SITE 12: note = D-threonine
PWVPSWMPPRHT

SEQ ID NO: 10 - protein, 9 aa, synthetic construct
  REGION 1..9: note = Circular
CRTIGPSVC

SEQ ID NO: 11 - protein, 30 aa, synthetic construct
YQQILTSMPSRNVIQISNDLENLRDLLHVL

SEQ ID NO: 12 - protein, 29 aa, synthetic construct
YTIWMPENPRPGTPCDIFTNSRGKRASNG

SEQ ID NO: 13 - protein, 16 aa, synthetic construct
  SITE 2: note = D-arginine
  SITE 3: note = D-glutamic acid
  SITE 4: note = D-isoleucine
  SITE 5: note = D-arginine
  SITE 6: note = D-threonine
  SITE 8: note = D-arginine
  SITE 9: note = D-alanine
  SITE 10: note = D-glutamic acid
  SITE 11: note = D-arginine
  SITE 12: note = D-tryptophan
  SITE 13: note = D-serine
  SITE 14: note = D-glutamic acid
  SITE 15: note = D-lysine
  SITE 16: note = D-phenylalanine
GREIRTGRAERWSEKF

SEQ ID NO: 14 - protein, 18 aa, synthetic construct
  SITE 18: note = Amidated
  SITE 1: note = Attached to position 11
  SITE 3: note = Attached to position 15
  SITE 11: note = Attached to position 1
  SITE 15: note = Attached to position 3
CNCKAPETALCARRCQQH

SEQ ID NO: 15 - protein, 8 aa, synthetic construct
  REGION 1..8: note = Circular
  SITE 1: note = Diaminopropionic acid present
KAPETALD

SEQ ID NO: 16 - protein, 13 aa, synthetic construct
HLNILSTLWKYRC

SEQ ID NO: 17 - protein, 7 aa, synthetic construct
  SITE 7: note = C- terminal amidation
  SITE 3: note = D-threonine
  SITE 7: note = O-beta-Glc present
GFTGFLS

SEQ ID NO: 18 - protein, 12 aa, synthetic construct
TGNYKALHPHNG

SEQ ID NO: 19 - protein, 11 aa, synthetic construct
  SITE 11: note = C- terminal amidation
YGRKKRRQRRR

SEQ ID NO: 20 - protein, 18 aa, synthetic construct
RGGRLSYSRRRFSTSTGR

SEQ ID NO: 21 - protein, 4 aa, synthetic construct
  REGION 1..4: note = Phenylproline
  SITE 4: note = C-terminal amidation
PPPP

SEQ ID NO: 22 - protein, 9 aa, synthetic construct
CRTIGPSVC

SEQ ID NO: 23 - protein, 21 aa, synthetic construct
LGGGGSGGGGSGGGGSGGGGS

SEQ ID NO: 24 - protein, 5 aa, synthetic construct
SGGGG

SEQ ID NO: 25 - protein, 7 aa, synthetic construct
SGGGGSG

SEQ ID NO: 26 - protein, 14 aa, synthetic construct
GAPLGGGGSGGGGS

SEQ ID NO: 27 - protein, 15 aa, synthetic construct
SGGSGGGGSGGGGSG

SEQ ID NO: 28 - protein, 4 aa, synthetic construct
SGGS

SEQ ID NO: 29 - protein, 10 aa, synthetic construct
GGSGGSGGSG

SEQ ID NO: 30 - protein, 22 aa, synthetic construct
ALCGGELVDTLQFVCGDRGFYF

SEQ ID NO: 31 - protein, 26 aa, synthetic construct
IVEECCFRSCDLALLETYCATPAKSE

SEQ ID NO: 32 - protein, 9 aa, synthetic construct
LRKLRKRLL

SEQ ID NO: 33 - protein, 6 aa, synthetic construct
CSGGGG

SEQ ID NO: 34 - protein, 8 aa, synthetic construct
SGGGGSGC

SEQ ID NO: 35 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 36 - protein, 24 aa, Homo sapiens
MGIPMGKSMLVLLTFLAFASCCIA

SEQ ID NO: 37 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 38 - protein, 91 aa, synthetic construct
MGIPMGKSMLVLLTFLAFASCCIAAYRPSETLCGGELVDTLQFVCGDRGFYFSRPASRVSRRSRGIVEECCFRSCDLALLETYCATPAKSE

SEQ ID NO: 39 - protein, 85 aa, synthetic construct
MGIPMGKSMLVLLTFLAFASCCIAALCGGELVDTLQFVCGDRGFYFSRPASRVSRRSRGIVEECCFRSCDLALLETYCATPAKSE

SEQ ID NO: 40 - protein, 74 aa, synthetic construct
MGIPMGKSMLVLLTFLAFASCCIAALCGGELVDTLQFVCGDRGFYFSGIVEECCFRSCDLALLETYCATPAKSE

SEQ ID NO: 41 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 42 - protein, 14 aa, Homo sapiens
MPPPRTGRGLLWLG

SEQ ID NO: 43 - RNA, 1653 nt, Homo sapiens
  source 1..1653: mol_type = mRNA
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 44 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 45 - RNA, 3566 nt, Homo sapiens
  source 1..3566: mol_type = mRNA
cgaccccggagtctccgcgggcggccagggcgcgcgtgcgcggaggttctcctcgtccgcccgttgttcagcgagggaggctctgcgcgtgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcag

SEQ ID NO: 46 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 47 - protein, 19 aa, Homo sapiens
MGLQACLLGLFALILSGKC

SEQ ID NO: 48 - RNA, 1692 nt, Homo sapiens
  source 1..1692: mol_type = mRNA
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctga

SEQ ID NO: 49 - protein, 4 aa, synthetic construct
SGGG

(19) United States
(12) Patent Application Publication
(10) Pub. No.: US 2026/0152730 A1
(43) Pub. Date: Jun. 4, 2026

(54) GENE THERAPY CONSTRUCTS FOR METABOLIC DISORDERS

(71) Applicant: Erasmus University Medical Center Rotterdam, Rotterdam (NL)

(72) Inventors: Wilhelmus Wenceslaus Matthias PIJNAPPEL, Vleuten (NL); Fabio CATALANO, Rotterdam (NL)

(21) Appl. No.: 19/126,215

(22) PCT Filed: Nov. 1, 2023
(86) PCT No.: PCT/NL2023/050576

§ 371 Date: Apr. 30, 2025

(30) Foreign Application Priority Data
Nov. 1, 2022 (EP) 22204936.3

(51) Int. Cl. C12N 9/16; A61K 38/00; A61K 48/00; A61P 3/00; C07K 14/65; C07K 14/775; C12N 15/86

(52) U.S. Cl. CPC C12N 9/16; A61K 48/005; A61P 3/00; C07K 14/65; C07K 14/775; C12N 15/86; A61K 38/00; C07K 2319/10; C12N 2740/15043; C12Y 301/06013

(57) ABSTRACT

The invention relates to nucleic acid molecules comprising a nucleotide sequence encoding a metabolic protein or a part thereof or a sequence having at least 90% sequence identity to said metabolic protein or part thereof, a human insulin-like growth factor II (IGFII) gene sequence, and a nucleotide sequence encoding at least one peptide that facilitates cellular uptake or transcytosis which is inserted at a location between the nucleotides encoding amino acids 28 and 42 of mature IGFII of said IGFII gene sequence. The invention further relates to related viral particles, fusion proteins and uses thereof.

### FIELD OF THE INVENTION

The invention relates to the field of gene therapy, in particular to gene delivery vehicles and method of gene therapy for treatment of metabolic disorders including lysosomal storage disorders.

### BACKGROUND OF THE INVENTION

Metabolic disorders are congenital errors of metabolism that affect the metabolism of macro- and micronutrients including carbohydrates, proteins, amino acids, lipids and fatty acids. Most metabolic disorders are caused by the genetic deficiency of an enzyme. Lysosomal storage disorders (LSDs) are metabolic disorders characterized by lysosomal dysfunction, most of which are inherited as autosomal recessive traits. Lysosomes are membrane-enclosed organelles that contain a variety of enzymes capable of breaking down all types of biological substances, i.e. proteins, nucleic acids, carbohydrates and lipids. Lysosomes serve both to degrade material taken up from outside the cell, including microorganisms, and to digest components of the cell itself. Many LSDs are caused by the absence or the deficiency of one or more lysosomal proteins (hydrolases, transport proteins, receptor molecules, ion pumps), in particular lysosomal hydrolases. Most LSDs have a progressive degenerative disease course. Their manifestations include lysosomal accumulation (“storage”) of non-degraded substrates, cell damage and tissue dysfunction resulting in morbidity and premature mortality.

Enzyme replacement therapy (ERT) has been developed for several LSDs including Pompe Disease and Hunter syndrome, in which recombinant human enzyme is administered intravenously. This treatment is aimed to increase the intracellular level of enzyme activity in affected cells and tissues and thereby reduce or prevent lysosomal accumulation and eventually symptoms of the disease.

Normally, missorted and therefore secreted lysosomal enzymes are redirected to the lysosomes through a receptor-mediated endocytosis mechanism via the CI-M6P/IGF2 receptor. This endogenous process is exploited in a number of other approaches for the treatment of LSDs and points at hematopoietic stem cells (HSCs) as the preferred target for ex vivo gene therapy. Since HSCs divide several times across the life span, long-term expression of the corrected gene is possible and vectors able to integrate into the host genome are preferable. Lentiviral vectors or site-specific gene editing approaches fulfil these requirements and could represent a therapeutic option for the treatment of LDSs via HSCs-mediated gene therapy. Gene corrected HSCs-derived cells, once transplanted, are able to synthetize and secrete the therapeutic protein which will be successively taken up by the target tissues, allowing cross correction of the disease pathology.

WO 2018/146473 describes a gene therapy strategy for mucopolysaccharidosis type II (MPSII) or Hunter syndrome, which is caused by mutation in the gene encoding iduronate 2-sulfatase (IDS gene). The construct contains an IDS gene sequence and a repeat of (a part of) the Apolipoprotein E (ApoEII) gene sequence.

WO 2008/136670, Van Til et al. (Blood. 2010 Jul. 1; 115(26):5329-37) and Wagemaker et al. (Mol Ther Methods Clin Dev. 2020 May 4; 17:1014-1025) describe lentiviral constructs containing the acid α-glucosidase encoding gene (GAA gene), that is deficient in Pompe disease, for transducing murine hematopoietic stem cells. Recently, Dogan et al. (https://doi.org/10.1101/2021.12.28.474352) described several constructs for hematopoietic stem cell gene therapy, among which untagged codon optimized GAA (GAAco) and GAA lacking the signal peptide tagged with several IGFII and modified IGFII tags.

ERT results in a variable clinical response (due to antibody formation to the recombinant enzyme and due to other unknown factors), is very invasive (4-6 hour infusion every 1-2 weeks), and does not provide a cure for the LSD.

Current lentiviral gene therapy is based on integration of the lentivirus into the genomic DNA. For safety reasons, the number of integrations per genome should be kept to a minimum of maximally ˜5 copies/genome or less while reaching sufficient therapeutic levels of the enzyme. Current gene therapy strategies suffer from insufficient therapeutic levels in all target tissues, i.e. peripheral tissues and the central and peripheral nervous system. In addition, undesired effects of the current epitope tags, such as the IGF2 tag, and antibody formation against the therapeutic enzyme are further safety concerns of current gene therapy strategies.

Hence, there remains a need in the art for gene therapy strategies with enhanced therapeutic efficacy and safety.

### SUMMARY OF THE INVENTION

It is an object of the present invention to overcome one or more problems that are associated with the above described therapies, in particular gene therapies, for treatment of metabolic disorders, in particular lysosomal storage disorders. It is a further object to provide nucleic acid molecules, gene therapy vectors and viral particles for gene therapy of such disorders.

In a first aspect, the invention therefore provides a nucleic acid molecule comprising:
a nucleotide sequence encoding a metabolic protein or a part thereof or a sequence having at least 90% sequence identity to said metabolic protein or part thereof,
a human insulin-like growth factor II (IGFII) gene sequence, and
a nucleotide sequence encoding at least one peptide that facilitates cellular uptake or transcytosis which is inserted at a location between the nucleotides encoding amino acids 28 and 42 of mature IGFII of said IGFII gene sequence.

Adjacent to the inserted sequence, which is located between the nucleotides encoding amino acids 28 and 42 of the mature IGFII of the mentioned IGFII gene sequence, optional flexible linker sequences are present. These linkers are designed to support the proper exposure of the inserted sequence. Surrounding these potential flexible linkers, cysteine residues are optionally present to support the formation of disulfide bonds, thereby ensuring the structural integrity of IGFII.

In a further aspect, the invention provides a nucleic acid molecule comprising a nucleotide sequence encoding a metabolic protein or a part thereof or a sequence having at least 90% sequence identity to said metabolic protein or part thereof, and a receptor associated protein (RAP) sequence, preferably a RAP tag, more preferably amino acids 251-262 of human RAP (EAKIEKHNHYQK; RAP12) or a repeat thereof, such as RAP12x2 (EAKIEKHNHYQKGEAKIEKHNHYQK).

In a further aspect, the invention provides a viral particle comprising a nucleic acid molecule according to the invention.

In a further aspect, the invention provides a fusion protein encoded by the nucleic acid molecule according to the invention.

In a further aspect, the invention provides a fusion protein comprising a metabolic protein or a part thereof or a sequence having at least 90% sequence identity to said metabolic protein or part thereof fused to a human insulin-like growth factor II (IGFII) sequence and at least one peptide that facilitates cellular uptake or transcytosis, wherein said metabolic protein or part thereof or sequence having at least 90% sequence identity to said metabolic protein or part thereof, human IGFII sequence and/or said at least one peptide are separated by one or more linking sequences.

In a further aspect, the invention provides a nucleic acid sequence encoding the fusion peptide according to the invention.

In a further aspect, the invention provides a cell population, preferably a hematopoietic stem cell (HSC) population, provided with a nucleic acid molecule, vector or viral particle according to the invention.

In a further aspect, the invention provides a nucleic acid molecule, viral particle, fusion protein or cell population according to the invention for use in a method for the treatment of a metabolic disorder, preferably a lysosomal storage disorder.

In a further aspect, the invention provides a method of treatment comprising administering nucleic acid molecule, viral particle, fusion protein or cell population according to the invention to an individual in need thereof.

In a further aspect, the invention provides a use of a nucleic acid molecule, viral particle, fusion protein or cell population according to the invention in the preparation of a medicament for the treatment of a metabolic disorder, preferably a lysosomal storage disorder.

### DETAILED DESCRIPTION

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (e.g. approximately 10, about 10) preferably means that the value may be the given value (e.g. 10), plus or minus 5% of the value (e.g. 10, plus or minus 5%), preferably plus or minus 1% of the value.

The use of the alternative (e.g., “or”) should be understood to mean either one, both, or any combination thereof of the alternatives.

As used herein, a nucleic acid molecule of the invention comprises a chain of nucleotides of any length, preferably DNA and/or RNA. In other embodiments a nucleic acid molecule or nucleic acid sequence of the invention comprises other kinds of nucleic acid structures such as for instance a DNA/RNA helix, peptide nucleic acid (PNA), locked nucleic acid (LNA) and/or a ribozyme. Hence, the term “nucleotide sequence” also encompasses a chain comprising non-natural nucleotides, modified nucleotides and/or non-nucleotide building blocks which exhibit the same function as natural nucleotides. The term nucleic acid molecule includes recombinant and synthetic nucleic acid molecules as well as nucleic acid molecules which are isolated, e.g. in part, from a natural source. In preferred embodiments of the present invention a nucleic acid molecule is DNA or RNA. It may be single stranded or double stranded. A skilled person is well capable of preparing nucleic acid molecules and vector of the invention, for instance using standard molecular cloning techniques as for instance described in Sambrook, J. and Russell, W., Molecular Cloning: A Laboratory Manual, Third Edition, Cold Spring Harbor Laboratory Press, Cold Spring Harbor (2001), which is incorporated herein by reference.

The term “peptide” refers to compounds comprising amino acids joined via peptide bonds. In particular, “peptide” to refers to small proteins containing up to 50 amino acid residues.

In amino acid sequences as defined or recited herein amino acids are denoted by single-letter symbols. These single-letter symbols and three-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine.

As used herein with respect to an amino acid sequence of a peptide, protein or fusion protein, the terms “N-terminal” and “C-terminal” refer to relative positions in the amino acid sequence toward the N-terminus and the C-terminus, respectively. “N-terminus” and “C-terminus” refer to the extreme amino and carboxyl ends of the polypeptide, respectively. “Immediately N-terminal” and “immediately C-terminal” refers to a position of a first amino acid sequence relative to a second amino acid sequence where the first and second amino acid sequences are covalently bound to provide a contiguous amino acid sequence.

The percentage of identity of an amino acid sequence or nucleic acid sequence, or the term “% sequence identity”, is defined herein as the percentage of residues of the full length of an amino acid sequence or nucleic acid sequence that is identical with the residues in a reference amino acid sequence or nucleic acid sequence after aligning the two sequences and introducing gaps, if necessary, to achieve the maximum percent identity. Methods and computer programs for the alignment are well known in the art, for example “Align 2”. Programs for determining nucleotide sequence identity are also well known in the art, for example, the BESTFIT, FASTA and GAP programs. These programs are readily utilized with the default parameters recommended by the manufacturer.

As used herein, the term “individual” encompasses humans. Preferably, a subject is a human, in particular a human suffering from a metabolic disorder, in particular a lysosomal storage disorder, more preferably Hunter syndrome, Pompe disease or CLN2. In some embodiments, the individual is a child, i.e. up to 18 years of age. In other embodiments, the individual is an adult, i.e. from 18 year of age.

The term “therapeutically effective amount,” as used herein, refers to an amount of an agent or composition being administered sufficient to relieve one or more of the symptoms of the disease or condition being treated to some extent. This can be a reduction or alleviation of symptoms, reduction or alleviation of causes of the disease or condition or any other desired therapeutic effect.

The term “treatment” refers to inhibiting the disease or condition, i.e., halting or reducing its development or at least one clinical symptom of the disease or disorder, and/or to relieving symptoms of the disease or condition. In some embodiments, treatment may be administered after one or more symptoms have developed. In other embodiments, treatment may be administered in the absence of symptoms. For example, treatment may be administered to a susceptible individual prior to the onset of symptoms (e.g., in light of genetic or other susceptibility factors). Treatment may also be continued after symptoms have resolved, for example to prevent or delay their recurrence.

The present inventors have developed a modular, tuneable fusion protein-encoding construct. The construct comprises a combination of two epitope tags. One of the epitope tags is an IGFII sequence and the other epitope tag is another tag, based on one or more peptides that facilitates cellular uptake or transcytosis, such as peptides that facilitate passage over the blood brain barrier (BBB). As demonstrated in the Examples herein, a suitable tuneable construct is a lentiviral construct of a lysosomal hydrolase, IDS in the examples, and a combination of the two different epitope tags. Multiple examples of the second epitope tag are included in the Examples herein, i.e. an ApoE2-derived tag, an ApoB-derived tag, a RAP12 tag, a leptin-30 tag and an angiopep2 tag. For all these tags, uptake of the fusion protein in the desired cells (see FIGS. 6 and 7), a reduction in lysosomal substrate accumulation (see e.g. FIGS. 12, 14 and 17), as well as the ability to bind the CI-M6P/IGFIIR (FIG. 19A), a reduced binding to the insulin receptor (FIG. 19B) and the ability to bind additional receptors (e.g. LRP-1) which are normally not bound by the IGFII tag, has been demonstrated.

The inventive principle underlying the constructs of the present invention is not limited to one specific lysosomal hydrolase, but it amenable to metabolic proteins in general, in particular metabolic enzymes of which a deficiency is associated with a metabolic disorder. As such, the constructs and other product are a modular system with three main components, 1) a metabolic protein sequence, 2) a human IGFII sequence and 3) a sequence for a second epitope tag. In particular components 1) and 3) can be varied. Or, alternatively, components 2) and 3) are present in an intermediate construct, wherein component 1), the metabolic protein sequence, can be varied.

The modular, tuneable constructs of the invention use a combination of three main components, 1) a metabolic protein sequence, 2) a human IGFII sequence and 3) a sequence for a second epitope tag, which thus are tuneable in that they modulate biodistribution of the relevant therapeutic protein for different diseases by selecting the relevant metabolic protein sequence. Secondly, by making use of a combination of two different epitope tags, two different mechanisms to increase the presence of the relevant metabolic protein at the target site are exploited. The IGFII tag allows for uptake of the encoded fusion protein via the cation independent mannose 6-phosphate or IGF2 receptor (CI-M6PR/IGF2R) by targeting the fusion protein to the lysosomes. In case of lysosomal hydrolases such as IDS and GAA, which already bind to the CI-M6PR via M6P present in these proteins, the IGFII tag increases lysosomal targeting because IGFII binds the same receptor with higher affinity. This is in particular advantageous for treatment of LSDs, because the different disorders are characterized by differences in the organs that are predominately affected. For instance, the main affected organs in Pompe disease include skeletal muscle, brain/central nervous system and heart, whereas the main affected organs in Hunter syndrome include lungs, heart, joints, connective tissue, and brain/central nervous system. In particular, the second epitope tag can be selected to target a different receptor than the CI-M6PR/IGF2R. This way, the uptake of the relevant metabolic protein, such as a lysosomal enzyme, is specific for the relevant organs that are affected by the particular LSDs, such as the brain and nervous system by making use of a further epitope that binds to a different receptor, in particular the LRP-1 receptor, and facilitates uptake in target cell or passage over the blood brain barrier. IGFII and the second tag that facilitates passage over the BBB, e.g. an ApoE-based tag, both bind to receptors expressed by the endothelial cells (ECs) that form the BBB. However, the amount of receptor expressed by the ECs of the BBB is limited, which is in particular for receptors expressed by human ECs (see Wang et al. 2020. Fluids and Barriers of the CNS; 17(47); doi.org/10.1186/s12987-020-00209-0). This limits the amount of therapeutic protein that can cross the BBB if only a single tag, such as the IGFII that binds to the CI-M6PR/IGF2R alone is used. By targeting multiple receptors, i.e. by using two or more different epitope tags that bind to different receptors, it now has become possible to proportionally increase the amount of therapeutic protein that can cross the BBB. This applies in particular if the first receptor is saturated so that the second, and optionally further, tag that is specific for a different receptor increases BBB uptake. The present inventors have shown that the constructs with a double epitope tag have an affinity for the receptor CI-M6PR/IGF2R that is comparable with the affinity of construct having only an IGFII tag (see FIG. 19A). This demonstrates that the double epitope tag does not negatively affect the binding this receptor, which is required for lysosomal uptake of lysosomal enzymes. Furthermore, it was also demonstrated that the affinity of the double epitope tag constructs for the insulin receptor is even reduced as compared to that of a construct having only an IGFII tag (see FIG. 19B). Because binding to the insulin receptor is undesired, this is an advantage of the double epitope tag constructs of the present invention. This is achieved while the constructs with the double tag have binding affinity to receptors other than those normally bound by the IGFII tag, such as the LRP-1 (FIGS. 8A and 8B). Additionally, the present inventors found that epitope tagging for BBB crossing does not always work when the tags are included in a lentiviral construct, a problem that is successfully addressed with the so-called indel constructs of the present invention. This is exemplified for the RAP12x2 tag in FIG. 8 herein. It is demonstrated that a RAP12x2 tag that is coupled to an IDS sequence does not efficiently bind to its target, the LRP1 receptor, in a functional ELISA assay (see FIG. 8A). When the RAP12x2 sequence is inserted into the IGFII sequence, (i.e. the IDS.IGF2indelRAP12x2 construct), it is able to exert its normal function, i.e. binding to the LRP1 receptor, and thus facilitates passage over the BBB (FIG. 8B). Indeed, it is generally known in the art that epitope tagging can interfere with the normal function of a protein to which it is coupled, as well as with the function of the tag itself due to steric hinderance of the tagged protein. Whether or not this happens is difficult to predict for a specific tag-protein combination. The present inventors show that insertion of a second epitope tag between amino acids 29 and 41 of the IGFII sequence provides a strategy to avoid non-functionality of tags e.g. due to steric hindrance. In particular, insertion of the second epitope tag in an IGFII sequence from which amino acids 30-40 or 29-41 are deleted, results in a favorable conformation of the second epitope tag, such that this tag is able to bind to its receptor. This could be supported by the presence of flexible linker sequences adjacent to the inserted sequence, which is located between the nucleotides encoding amino acids 28 and 42 of the mature IGFII of the mentioned IGFII gene sequence, which result in a favourable exposure of the inserted tag. In addition, a cysteine residue can be added at the N- and C-terminus of both flexible linkers to support the correct conformation of the IGFII tag.

Hence, the constructs of the present invention can be tuned for specific metabolic disorders, in particular specific LSDs, and tissue specific targeting and uptake by selecting an advantageous combination of the metabolic protein, such a lysosomal hydrolase, and second epitope tag. In particular, the IGFII sequence is a fixed component of the modular construct, while the second epitope is aligned with the specific metabolic protein and, consequently, with the specific disorder. The present inventors have shown that good results are obtained when the second epitope tag is inserted within the IGFII sequence, in particular at a location in the IGFII that is required for efficient binding to insulin receptor (see U.S. Pat. No. 9,469,683B2). This way, undesired side effects due to binding of the encoded fusion protein to the insulin receptor are avoided. The present inventors have previously shown (unpublished data, e.g. Dutch patent application NL 2031676) that with a vector comprising an IGFII sequence with a deletion of or within the nucleotide sequence encoding the insulin binding receptor, efficacy in the brain is also achieved. This is surprising, as the insulin receptor was believed to be important for crossing the blood brain barrier, because the insulin receptor has been reported to mediate transcytosis in vivo, while there have been no such reports to date for the CI-M6PR. This can also be deduced from the lack of efficacy of intravenously applied enzyme replacement therapy (which targets the CI-M6PR) in the brain in lysosomal disorders.

In a first aspect, the invention therefore provides a nucleic acid molecule comprising:
a nucleotide sequence encoding a metabolic protein or a part thereof or a sequence having at least 90% sequence identity to said metabolic protein or part thereof,
a human insulin-like growth factor II (IGFII) gene sequence, and
a nucleotide sequence encoding at least one peptide that facilitates cellular uptake or transcytosis.

In some aspects, a nucleic acid molecule of the invention comprises a human insulin-like growth factor II (IGFII or IGF2) gene sequence. FIG. 1 shows the sequence of human mature IGFII, including its signal peptide. IGFII is synthesized as a pro-protein containing 180 amino acids which is processed ultimately into a 67-amino acid bioactive IGFII protein, which is referred to as mature IGFII. Firstly, a signal peptide of 24 amino acids is removed from the N terminus, generating pro-IGFII (156 amino acids), followed by cleavage of pro-IGFII into a 104-amino acid protein product [IGFII (1-104)]. Subsequent endoproteolyzation generates IGFII (1-87) and the mature IGFII protein consisting of 67 amino acids.

In preferred embodiments, the IGFII gene sequence comprises a sequence encoding amino acids 8-28 and 42-67 of human mature IGFII, as shown in FIG. 1, or a sequence having at least 70% sequence identity thereto, preferably at least 80% sequence identity thereto, more preferably at least 90% sequence identity thereto, more preferably at least 95% sequence identity thereto. In some preferred embodiments, the IGFII gene sequence comprises a sequence encoding amino acids 8-29 and 41-67 of human mature IGFII, as shown in FIG. 1, or a sequence having at least 70% sequence identity thereto, preferably at least 80% sequence identity thereto, more preferably at least 90% sequence identity thereto, more preferably at least 95% sequence identity thereto.

In preferred embodiments, amino acids 2-7 are omitted as this results in a significant decrease in affinity for the human IGF-I receptor and increase for the IGFII receptor (Hashimoto et al., J. Biol. Chem. 270(30):18013-8(1995). Hence, in further preferred embodiments, the IGFII gene sequence comprises a sequence encoding amino acids 1, 8-28 and 42-67 or 1, 8-29 and 41-67 of human mature IGFII, as shown in FIG. 1, or a sequence having at least 70% sequence identity thereto, preferably at least 80% sequence identity thereto, more preferably at least 90% sequence identity thereto, more preferably at least 95% sequence identity thereto.

In preferred embodiments, the IGFII sequence encodes an IGFII amino acid sequence which has a mutation that reduces binding affinity for the insulin receptor as compared to the wild-type human IGF-II. In some preferred embodiments, the human IGFII gene sequence comprises a mutation within the nucleotide region encoding amino acids 29-41 of IGFII, preferably a deletion within the nucleotide region encoding amino acids 29-41 of IGFII. This region of the IGFII sequence comprises the insulin receptor binding domain. Hence, the IGFII sequence preferably is mutated such that binding of the fusion protein to the insulin receptor is reduced with at least 50% or abolished. In preferred embodiments, the IGFII gene sequence comprises a deletion of the nucleotides encoding at least amino acids 29-41, 30-41, 31-41, 32-41, 33-41, 34-41, 35-41, 36-41, 37-41, 29-40, 30-40, 31-40, 32-40, 33-40, 34-40, 35-40, 36-40, 37-40, 29-39, 30-39, 31-39, 32-39, 33-39, 34-39, 35-39, 36-39, 29-38, 30-38, 31-38, 32-38, 33-38, 34-38, 35-38, 36-38, 29-37, 30-37, 31-37, 32-37, 33-37, 34-37, 35-37, 29-36, 30-36, 31-36, 32-36, 33-36, 34-36, 29-35, 30-35, 31-35, 32-35, 33-35, 29-34, 30-34, 31-34, 30-34, 29-33, 30-33, 31-33, 29-32 or 30-32 of human mature IGFII as shown in FIG. 1.

In preferred embodiments, the IGFII gene sequence comprises a deletion of the nucleotides encoding amino acids 30-40 of human mature IGFII. Hence, in preferred embodiments, the IGFII gene sequence comprises a nucleotide sequence encoding amino acids 1, 8-29 and 41-67 of mature IGFII, as shown in FIG. 1.

In preferred embodiments, the IGFII gene sequence comprises a deletion of the nucleotides encoding amino acids 29-41 of human mature IGFII. Hence, in preferred embodiments, the IGFII gene sequence comprises a nucleotide sequence encoding amino acids 1, 8-28 and 42-67 of mature IGFII, as shown in FIG. 1.

In some preferred embodiments, the IGFII gene sequence consists of a nucleotide sequence encoding amino acids 1 and 8-28 and 42-67 of human mature IGFII, as shown in FIG. 1.

In some preferred embodiments, the IGFII gene sequence consists of a nucleotide sequence encoding amino acids 1 and 8-29 and 41-67 of human mature IGFII, as shown in FIG. 1.

Further suitable IGFII mutants with reduced insulin receptor binding are described in WO 2009/137721, which is incorporated herein by reference.

As detailed herein below, the nucleotide sequence encoding at least one peptide that facilitates cellular uptake or transcytosis, preferably at least one peptide that facilitates passage over the BBB, is preferably inserted at a location between the nucleotides encoding of amino acids 28 and 42 of human mature IGFII of said IGFII gene sequence. If the IGFII sequence comprises a mutation within the nucleotide region encoding amino acids 29-41 of IGFII as detailed herein above, the nucleotide sequence encoding at least one peptide that facilitates cellular uptake or transcytosis, preferably at least one peptide that facilitates passage over the BBB, is preferably inserted at the location of the deleted amino acids of the IGFII sequence.

In other embodiments, the IGFII gene sequence comprises a nucleotide sequence encoding the IGFII signal peptide and amino acids 8-67, preferably 1 and 8-67 of IGFII, as shown in FIG. 1, or an amino acid sequence which has at least 70%, preferably at least 80%, more preferably at least 90%, more preferably at least 95%, sequence identity thereto. In some embodiments, the IGFII gene sequence encodes human, mature IGFII, the sequence of which is shown in FIG. 1 (i.e. amino acids 1-67), or an amino acid sequence which has at least 70%, preferably at least 80%, more preferably at least 90%, more preferably at least 95%, sequence identity thereto. In some embodiments, the IGFII gene sequence consists of a nucleotide sequence encoding amino acids 1 and 8-67 of human mature IGFII, as shown in FIG. 1.

In some embodiments, the IGFII gene sequence comprises a nucleotide sequence encoding the human IGFII signal peptide, indicated as amino acids −24 to −1 in FIG. 1 (MGIPMGKSMLVLLTFLAFASCCIA), or a sequence having at least 90% sequence identity therewith. Preferably, the IGFII gene sequence comprises a nucleotide sequence encoding the human IGFII signal peptide, indicated as amino acids −24 to −1 in FIG. 1.

The use of a nucleic acid molecule or vector comprising such IGFII sequence with a deletion of or within the nucleotide sequence encoding the insulin binding receptor, preferably leaves binding to the IGFII/mannose 6 phosphate receptor (IGFIIR/M6PR) intact. It was found that the IGFII gene sequence with a deletion of or within the nucleotide sequence encoding the insulin binding receptor domain improved safety with respect to glucose metabolism, which is of clinical relevance.

A nucleic acid molecule of the invention comprises a nucleotide sequence encoding at least one peptide that facilitates cellular uptake or transcytosis. As used herein “facilitates cellular uptake or transcytosis” means that a protein or polypeptide that is expressed from the nucleic acid molecule of the invention has increased cellular uptake or transcytosis if the at least one peptide is present in the protein or polypeptide as compared to a protein or polypeptide that is lacking the at least one peptide but otherwise identical. The peptide that facilitates cellular uptake or transcytosis is not IGFII or derived from IGFII.

As used herein “cellular uptake” refer to the internalization of a molecule or compound into a cell, in particular of a proteinaceous compound encoded by a nucleic acid molecule of the invention. As used herein “transcytosis” refers to a mechanism for transcellular, vesicular transport in which a cell encloses a molecule or compound in vesicles formed by the cell membrane and transfers the vesicle across the cell to eject the material through the opposite cell membrane.

Transcytosis is in particular transcellular, vesicular transport across endothelial cells. In a preferred embodiment, transcytosis is transcellular, vesicular transport across the blood brain barrier (BBB).

In preferred embodiments, the at least one peptide that facilitates cellular uptake or transcytosis has a length of at most 100 amino acids. More preferably, this peptide has length of at most 90 amino acids, more preferably at most 80 amino acids, more preferably at most 70 amino acids, more preferably at most 60 amino acids, more preferably at most 50 amino acids, more preferably at most 40 amino acids, such as at most 39, at most 40, at most 41, at most 42 at most 43, at most 44 or at most 45 amino acids.

In preferred embodiments, the at least one peptide that facilitates cellular uptake or transcytosis is at least one peptide that facilitates passage over the BBB. As used herein “blood brain barrier” and “BBB” refer to a functional barrier between the blood circulation and the brain and spinal cord, that strictly controls transfer of substances from blood to neural tissues, and which is mainly formed by (cerebrovascular) endothelial cells. As used herein “facilitates passage over the blood brain barrier” means that passage over the BBB of a protein or polypeptide that is expressed from the nucleic acid molecule of the invention is increased if the at least one peptide that facilitates passage over the BBB is present in the protein or polypeptide as compared to a protein or polypeptide that is lacking the at least one peptide that facilitates passage over the BBB but otherwise identical.

Peptides that facilitate passage over the BBB are well known in the art. Preferred, but non-limiting peptides include: Angiopep-2 (TFFYGGSRGKRNNFKTEEY-OH); ApoB (3371-3409) (SSVIDALQYKLEGTTRLTRK-RGLKLATALSLSNKFVEGS); ApoE (159-167) 2 ((LRKLRKRLL) 2); Peptide-22 (Ac-C(&)MPRLRGC(&)-NH₂; & indicates cyclic peptide whereby C(&) and C(&) are attached); THR (THRPPMWSPVWP-NH₂); THR retro-enantio (pwvpswmpprht-NH₂); CRT (C(&)RTIGPSVC(&); & indicates cyclic peptide whereby C(&) and C(&) are attached); Leptin30 (YQQILTSMPSRNVIQISND-LENLRDLLHVL); RVG29 (YTIWMPENPRPGTPCDIFT-NSRGKRASNG-OH); ^{D}CDS (GreirtGraerwsekf-OH); Apamin (C(&₁)NC(&₂)KAPETALC(&₁)-AR-RC(&₂)QQH-NH₂; & indicates cyclic peptide, whereby C(&1) and C(&1) are attached and C(&2) and C(&2) are attached); MiniAp-4 ([Dap](&)KAPETALD(&); whereby Dap=diaminopropionic acid and & indicates cyclic peptide, whereby & and & are attached); GSH (γ-L-glutamyl-CG-OH); G23 (HLNILSTLWKYRC); g⁷ (GFtGFLS(O-β-Glc)-NH₂), TGN (TGNYKALHPHNG), TAT (47-57) (YGRKKRRQRRR-NH₂), SynB1 (RGGRLSYSRRRFSTSTGR), and PhPro ((Phenylproline)₄-NH₂).

In preferred embodiments, the peptide that facilitates cellular uptake or transcytosis, in particular that facilitate passage over the BBB, binds to a receptor that is present on endothelial cells and/or that is involved in transcytosis. Examples of such receptors are the insulin receptor, in particular M6PR (mannose-6-phosphate receptor), leptin receptor, transferrin receptor and low density lipoprotein receptor (LRP). For example, the peptide contains a receptor binding domain of Apolipoproteins (Apo) A, B or E, receptor-associated protein (RAP), transferrin (Tf), lactotransferrin, melanotransferrin (p97), or leptin. Hence, in a preferred embodiment, the peptide comprises a receptor binding domain of ApoA, ApoB, ApoE, RAP, transferrin, lactotransferrin, melanotransferrin (p97), or leptin. Similarly, the nucleotide sequence encoding at least one peptide that facilitates cellular uptake or transcytosis, in particular encoding at least one peptide that facilitates passage over the BBB, encodes a receptor binding domain of ApoA, ApoB, ApoE, RAP, transferrin, lactotransferrin, melanotransferrin (p97), or leptin, or a combination and/or repeat of any of these domains.

In further preferred embodiments, the peptide that facilitates passage over the BBB comprises a low density lipoprotein receptor-related protein 1 (LRP1) binding domain. In preferred embodiments, the peptide that facilitates passage over the BBB comprising a LRP1 binding domain has a length of at most 100 amino acids. More preferably, this peptide has length of at most 90 amino acids, more preferably at most 80 amino acids, more preferably at most 70 amino acids, more preferably at most 60 amino acids, more preferably at most 50 amino acids, more preferably at most 40 amino acids, such as at most 39, at most 40, at most 41, at most 42 at most 43, at most 44 or at most 45 amino acids.

In further preferred embodiments, the nucleotide sequence encoding at least one peptide that facilitates cellular uptake or transcytosis encodes a receptor binding domain of human apolipoprotein E2 (ApoE2), human apolipoprotein B (ApoB), human receptor associated protein (RAP), such as RAP12 (EAKIEKHNHYQK) or RAP12x2 (EAKIEKHNHYQKGEAKIEKHNHYQK), or human leptin, or encodes CRP peptide (CRTIGPSVC) or Angiopep-2 (TFFYGGSRGKRNNFKTEEY), or a combination and/or repeat of any of these domains and sequences. Preferably, the peptide has a length of at most 100 amino acids. More preferably, this peptide has length of at most 90 amino acids, more preferably at most 80 amino acids, more preferably at most 70 amino acids, more preferably at most 60 amino acids, more preferably at most 50 amino acids, more preferably at most 40 amino acids, such as at most 39, at most 40, at most 41, at most 42 at most 43, at most 44 or at most 45 amino acids.

In further preferred embodiments, the peptide that facilitates passage over the BBB comprises or consists of a receptor binding domain of human apolipoprotein E2 (ApoE2), human apolipoprotein B (ApoB), human receptor associated protein (RAP), or human leptin, or comprises CRP peptide (CRTIGPSVC) or Angiopep-2 (TFFYGGSRGKRNNFKTEEY), or a combination and/or repeat of any of these domains and sequences. Preferably, the peptide has a length of at most 100 amino acids. More preferably, this peptide has length of at most 90 amino acids, more preferably at most 80 amino acids, more preferably at most 70 amino acids, more preferably at most 60 amino acids. In further preferred embodiments, the peptide has a length of at most 50 amino acids, more preferably at most 40 amino acids.

In further preferred embodiments, the nucleotide sequence encoding at least one peptide that facilitates cellular uptake or transcytosis encodes amino acids 141-149 of human ApoE2 (LRKLRKRLL), amino acids 3371-3409 of human ApoB (SSVIDALQYKLEGTTRLTRKRGLKLATALSLSNKFVEGS), amino acids 251-262 of human RAP (EAKIEKHNHYQK; RAP12), amino acids 61-90 of human leptin (YQQILTSMPSRNVIQISNDLENLRDLLHVL), or a combination and/or repeat of any of these sequences. Preferably, the peptide has a length of at most 100 amino acids. More preferably, this peptide has length of at most 90 amino acids, more preferably at most 80 amino acids, more preferably at most 70 amino acids, more preferably at most 60 amino acids. In further preferred embodiments, the peptide has a length of at most 50 amino acids, more preferably at most 40 amino acids.

In further preferred embodiments, the peptide that facilitates passage over the BBB comprises or consists of amino acids 141-149 of human ApoE2 (LRKLRKRLL), amino acids 3371-3409 of human ApoB (SSVIDALQYKLEGTTRLTRKRGLKLATALSLSNKFVEGS), amino acids 251-262 of human RAP (EAKIEKHNHYQK), amino acids 61-90 of human leptin (YQQILTSMPSRNVIQISNDLENLRDLLHVL), or a combination and/or repeat of any of these sequences. Preferably, the peptide has a length of at most 100 amino acids. More preferably, this peptide has length of at most 90 amino acids, more preferably at most 80 amino acids, more preferably at most 70 amino acids, more preferably at most 60 amino acids. In further preferred embodiments, the peptide has a length of at most 50 amino acids, more preferably at most 40 amino acids.

In further preferred embodiments, the peptide that facilitates passage over the BBB comprises or consists of amino acids 141-149 of human ApoE2 (LRKLRKRLL). Preferably, the peptide has a length of at most 100 amino acids. More preferably, this peptide has length of at most 90 amino acids, more preferably at most 80 amino acids, more preferably at most 70 amino acids, more preferably at most 60 amino acids. In further preferred embodiments, the peptide has a length of at most 50 amino acids, more preferably at most 40 amino acids. In further preferred embodiments, the peptide comprising amino acids 141-149 of human ApoE2 (LRKLRKRLL) has a length of at most 30, more preferably at most 25, more preferably at most 20, more preferably at most 15, more preferably at most 12 amino acids.

In further preferred embodiments, the peptide that facilitates passage over the BBB comprises or consists of a repeat of amino acids 141-149 of human ApoE2 ((LRKLRKRLL) 2). Preferably, the peptide has a length of at most 100 amino acids. More preferably, this peptide has length of at most 90 amino acids, more preferably at most 80 amino acids, more preferably at most 70 amino acids, more preferably at most 60 amino acids. In further preferred embodiments, the peptide has a length of at most 50 amino acids, more preferably at most 40 amino acids.

In further preferred embodiments, the peptide that facilitates passage over the BBB comprises or consists of amino acids 3371-3409 of human ApoB (SSVIDALQYKLEGTTRLTRKRGLKLATALSLSNKFVEGS).

In further preferred embodiments, the peptide that facilitates passage over the BBB comprises or consists of amino acids 61-90 of human leptin (YQQILTSMPSRNVIQISNDLENLRDLLHVL). Preferably, the peptide has a length of at most 100 amino acids. More preferably, this peptide has length of at most 90 amino acids, more preferably at most 80 amino acids, more preferably at most 70 amino acids, more preferably at most 60 amino acids. In further preferred embodiments, the peptide has a length of at most 50 amino acids, more preferably at most 40 amino acids. In further preferred embodiments, the peptide comprising amino acids 61-90 of human leptin (YQQILTSMPSRNVIQISNDLENLRDLLHVL) has a length of at most 35 amino acids.

In further preferred embodiments, the peptide that facilitates passage over the BBB comprises or consists of a repeat of amino acids 251-262 of human RAP (EAKIEKHNHYQKGEAKIEKHNHYQK; RAP12x2). Preferably, the peptide has a length of at most 100 amino acids. More preferably, this peptide has length of at most 90 amino acids, more preferably at most 80 amino acids, more preferably at most 70 amino acids, more preferably at most 60 amino acids. In further preferred embodiments, the peptide has a length of at most 50 amino acids, more preferably at most 40 amino acids. In further preferred embodiments, the peptide comprising a repeat of amino acids 251-262 of human RAP (EAKIEKHNHYQKGEAKIEKHNHYQK; RAP12x2) has a length of at most 35, more preferably at most 30 amino acids.

In some aspects, the invention provides a nucleic acid molecule comprising a nucleotide sequence encoding a metabolic protein or a part thereof or a sequence having at least 90% sequence identity to said metabolic protein or part thereof, and a receptor associated protein (RAP) sequence, preferably a RAP tag, more preferably amino acids 251-262 of human RAP (EAKIEKHNHYQK; RAP12) or a repeat thereof, such as RAP12x2 (EAKIEKHNHYQKGEAKIEKHNHYQK).

A nucleic acid molecule of the invention comprises a nucleotide sequence encoding a metabolic protein or a part thereof or a sequence having at least 90% sequence identity to said metabolic protein or part thereof. In particular, said metabolic protein is a protein that is associated with a metabolic disorder. E.g. the metabolic protein is a protein that is absent, mutated, reduced or otherwise impaired and thereby causes or contributes to the metabolic disorder. The aim of the nucleic acid molecule of the invention is to restore the expression, level and/or function of the metabolic protein.

A nucleic acid molecule of the invention comprises a nucleotide sequence encoding a metabolic protein can suitable be used to restore the expression, level and/or function of any metabolic protein. In preferred embodiments, the metabolic protein is a metabolic enzyme. In other preferred embodiments, the metabolic protein is a lysosomal protein. As used herein “lysosomal protein” refers to a protein that is present and functionally active in the lysosome. In further preferred embodiments, the metabolic protein is a lysosomal enzyme. As used herein a “lysosomal enzyme” refers to an enzyme present and functionally active in lysosomes, in particular in the degradation of extracellular material or obsolete components of the cell. There are over 50 known, inherited disorders where a link has been established between the disorder and mutation(s) in a gene coding for a lysosomal protein, in particular lysosomal enzyme. Such mutations lead to a deficiency or functional impairment of the lysosomal protein. These disorders are referred to as lysosomal storage disorders (LSDs) and are characterized by a build-up of the metabolite or metabolites that cannot or are insufficiently degraded resulting from the deficiency or functional impairment of the lysosomal protein.

In preferred embodiments, a nucleic acid molecule of the invention comprises a nucleotide sequence encoding metabolic protein, preferably lysosomal protein, more preferably lysosomal enzyme, more preferably lysosomal hydrolase, or a part thereof or a sequence having at least 95% sequence identity to said metabolic protein or part thereof, more preferably at least 96%, more preferably at least 97%, more preferably at least 98%, more preferably at least 99%, sequence identity with the metabolic protein, preferably lysosomal protein, more preferably lysosomal enzyme, more preferably lysosomal hydrolase, or a part thereof.

As used herein a “part” of a metabolic protein, lysosomal protein or lysosomal enzyme refers to an active part thereof, in particular having the same activity as the metabolic protein, lysosomal protein or lysosomal enzyme from which the part is derived, although not necessarily at the same level. In particular, a part of a lysosomal enzyme is an enzymatically active part thereof. As another examples, a part of a lysosomal hydrolase is an enzymatically active part thereof.

Examples of lysosomal proteins include lysosomal structural proteins, lysosomal membrane proteins and soluble lysosomal proteins, the latter including lysosomal enzymes. In a preferred embodiment, a nucleic acid molecule of the invention comprises a nucleotide sequence encoding a lysosomal enzyme or a part thereof or a sequence having at least 90% sequence identity to said lysosomal enzyme or part thereof. In further preferred embodiments, the lysosomal enzyme is a lysosomal hydrolase. In preferred embodiments, the lysosomal enzyme or lysosomal hydrolase is selected from the group consisting of human GLA (α-galactosidase A; Fabry disease), ASAH1 (acid ceramidase; Farber lipogranulomatosis), GBA (glucocerebrosidase; Gaucher disease), GLB1 (beta-galactosidase; GM1 gangliosidosis), HEXA (β-hexosaminidase A; GM2 gangliosidosis or Tay-Sachs disease), GM2A (GM2 activator; GM2 gangliosidosis or GM2 activator deficiency), GALC (galactocerebrosidase; Krabbe disease), ARSA (Arylsulfatase A; Metachromatic leukodystrophy), PSAP (saposin B; Metachromatic leukodystrophy), SMPD1 (sphingomyelin phosphodiesterase 1; Niemann-Pick A/B), LIPA (lysosomal acid lipase; Acid lipase deficiency: Wolman disease and cholesterol ester storage disease), IDUA (alpha-L-iduronidase; PMS I or Hurler syndrome), IDS (iduronate-2-sulfatase; PMS II or Hunter syndrome), SGSH (N-sulfoglucosamine sulfohydrolase; PMS IIIA), NAGLU (α-N-acetylglucosaminidase; PMS IIIB), HGSNAT (heparan-alpha-glucosaminide N-acetyltransferase; PMS IIIC), GNS (N-Acetylglucosamine-6-Sulfatase; PMS IIID), GALNS (N-acetylgalactosamine 6 sulfate sulfatase; PMS IVA), GLB1 (beta-galactosidase-1; PMS IVB), ARSB (arylsulfatase B; PMS VI), GUSB (β-glucuronidase; PMS VII), HYAL1 (hyaluronoglucosaminidase-1; PMS IX), SUMF1 (sulfatase-modifying factor-1; Multiple sulfatase deficiency), GNPTAB (N-acetylglucosamine-1-phosphotransferase; Mucolipidosis II α/β, I-cell disease and pseudo-Hurler polydystrophy), GNPTG (N-acetylglucosamine-1-phosphotransferase gamma subunit; Mucolipidosis III γ, variant pseudo-Hurler polydystrophy), MAN2B1 (alpha-mannosidase; α-Mannosidosis), MANBA (beta-mannosidase; β-Mannosidosis), FUCA1 (alpha-L-fucosidase; Fucosidosis), AGA (aspartylglucosamidase; Aspartylglucosaminuria), NAGA (alpha-N-acetylgalactosaminidase; Schindler disease), NEU1 (neuraminidase-1; Sialidosis type I and II), CTSA (protective protein/cathepsin A; Galactosialidosis), HPS1 (Hermansky-Pudlak syndrome 1 protein; Hermanski-Pudlak disease type 1), AP3BA (beta-3A subunit of the heterotetrameric AP3 complex; HPS2 or Hermanski-Pudlak disease type 2), HPS3 (Hermansky-Pudlak syndrome 3 protein; Hermanski-Pudlak disease type 3), HPS4 (Hermansky-Pudlak syndrome 4 protein; Hermanski-Pudlak disease type 4), HPS5 (Hermansky-Pudlak syndrome 5 protein; Hermanski-Pudlak disease type 5), HPS6 (Hermansky-Pudlak syndrome 6 protein; Hermanski-Pudlak disease type 6), HPS7 (Hermansky-Pudlak syndrome 7 protein; Hermanski-Pudlak disease type 7), BLOC1S3 (Biogenesis Of Lysosomal Organelles Complex 1 Subunit 3; HPS8 or Hermanski-Pudlak disease type 8), HPS9 (Hermansky-Pudlak syndrome 9 protein; Hermanski-Pudlak disease type 9), MYO5A (myosin Va; Griscelli syndrome 1), RAB27A (Rab GTPase family 27A; Griscelli syndrome 2), LYST (lysosomal trafficking regulator; Chédiak-Higashi disease), GAA (acid alpha glucosidase; Pompe disease), PPT1 (palmitoyl-protein thioesterase-1; Neuronal ceroid lipofuscinoses (CLN) 1), TPP1 (tripeptidyl peptidase 1; CLN2), CLN3 (ceroid-lipofuscinosis neuronal protein 3; CLN3), DNAJC5 (DnaJ homolog subfamily C member 5; CLN4), CLN5 (ceroid-lipofuscinosis neuronal protein 5; CLN5), CLN6 (ceroid-lipofuscinosis neuronal protein 6; CLN6), MFSD8 (ceroid-lipofuscinosis neuronal protein 7; CLN7), CLN8 (ceroid-lipofuscinosis neuronal protein 8; CLN8), CTSD (cathepsin D; CLN10), GRN (progranulin; CLN11), ATP13A2 (ATPase cation transporting 13A2; CLN12), CTSF (cathepsin F; CLN13), and KCTD7 (potassium channel tetramerization domain containing 7; CLN14). The LSD that is associated with deficiency of the relevant lysosomal enzyme is indicated between brackets.

In preferred embodiments, the lysosomal enzyme or lysosomal hydrolase is selected from the group consisting of human iduronate-2-sulfatase (IDS), acid alpha glucosidase (GAA), and tripeptidyl peptidase 1 (TPP1).

In some preferred embodiments, a nucleic acid molecule of the invention comprises a nucleotide sequence encoding amino acids 1-550 or 26-550 of human IDS or an enzymatically active part thereof or a sequence having at least 90%, preferably at least 95%, more preferably at least 98%, sequence identity to amino acids 1-550 or 26-550 of human IDS or an enzymatically active part thereof. In some preferred embodiments, a nucleic acid molecule of the invention comprises a nucleotide sequence encoding amino acids 1-550 or 26-550 of human IDS or an enzymatically active part thereof.

In some preferred embodiments, a nucleic acid molecule of the invention comprises a nucleotide sequence encoding amino acids 70-952, 28-952 or 1-952 of human GAA or an enzymatically active part thereof or a sequence having at least 90%, preferably at least 95%, more preferably at least 98%, sequence identity to amino acids 70-952, 28-952 or 1-952 of GAA or an enzymatically active part thereof. In some preferred embodiments, a nucleic acid molecule of the invention comprises a nucleotide sequence encoding human amino acids 70-952, 28-952 or 1-952 of GAA or an enzymatically active part thereof.

In some preferred embodiments, a nucleic acid molecule of the invention comprises a nucleotide sequence encoding human amino acids 1-563 or 20-563 of TPP1 or an enzymatically active part thereof or a sequence having at least 90%, preferably at least 95%, more preferably at least 98%, sequence identity to amino acids 1-563 or 20-563 of TPP1 or an enzymatically active part thereof. In some preferred embodiments, a nucleic acid molecule of the invention comprises a nucleotide sequence encoding human amino acids 1-563 or 20-563 of TPP1 or an enzymatically active part thereof.

Hunter syndrome or mucopolysaccharidosis type II (MPS II) is lysosomal storage disorder caused by mutations in the IDS gene. These mutations result in deficiency of the iduronate-2-sulfatase enzyme. IDS belongs to the sulfatase family of enzymes and catalyses hydrolysis of the C2-sulfate ester bond at the non-reducing end of 2-O-sulfo-α-1-iduronic acid residues in dermatan sulfate and heparan sulfate. Deficiency of IDS consequently results in lysosomal accumulation of dermatan sulfate (DS) and heparan sulfate (HS). Symptoms of Hunter syndrome are not present at birth, but typically begin around ages 2 to 4. Clinical symptoms of Hunter syndrome range from mild to severe and mostly present in the lungs, heart, joints, connective tissue, and brain and nervous system. Current treatment approaches for Hunter syndrome are tailored to specific patients and include enzyme replacement therapy (ERT) and symptom management.

The human IDS amino acid sequence encoded by the human IDS gene is shown in FIG. 2A. This sequence contains a signal peptide (amino acids 1-25). The human IDS cDNA sequence, including the sequence encoding the signal peptide is shown in FIG. 2B.

In some preferred embodiments, the metabolic protein is iduronate-2-sulfatase (IDS). In preferred embodiments, the nucleic acid molecule of the invention comprises a nucleotide sequence encoding an amino acid sequence comprising a sequence having at least 90% sequence identity with amino acids 26-550 of human IDS. In further preferred embodiments, said nucleotide sequence encodes amino acids 26-550 of human IDS. In further preferred embodiments, a nucleotide sequence encoding the IDS signal peptide is present. I.e. the nucleic acid molecule of the invention comprises a nucleotide sequence encoding an amino acid sequence comprising a sequence having at least 90% sequence identity with amino acids 1-550 of human IDS. Alternatively, another signal peptide is present, such as the IGFII signal peptide, which is shown in FIG. 1.

In some preferred embodiments, a nucleic acid molecule of the invention comprises a nucleotide sequence encoding amino acids 26-550 of human IDS, or an amino acid sequence having at least 95%, more preferably at least 96%, more preferably at least 97%, more preferably at least 98%, more preferably at least 99%, sequence identity with amino acids 26-550 of human IDS, or an amino acid sequence comprising or consisting of amino acids 26-550 of human IDS.

In some preferred embodiments, a nucleic acid molecule of the invention comprises a nucleotide sequence encoding amino acids 1-550 of human IDS, or an amino acid sequence having at least 95%, more preferably at least 96%, more preferably at least 97%, more preferably at least 98%, more preferably at least 99%, sequence identity with amino acids 1-550 of human IDS, or an amino acid sequence comprising or consisting of amino acids 1-550 of human IDS.

In some preferred embodiments, the variation in amino acid sequence is only in amino acids 26-550. I.e. in some preferred embodiments, the nucleic acid molecule encodes an amino acid sequence having at least 90%, more preferably at least 95%, more preferably at least 96%, more preferably at least 97%, more preferably at least 98%, more preferably at least 99%, sequence identity with amino acids 26-550 of human IDS and encodes amino acids 1-25 of human IDS.

In one preferred embodiment, the nucleotide sequence encoding amino acids 26-550 of IDS comprises or consists of nucleotides 245-1822 of the human IDS cDNA as shown in FIG. 2B. In another preferred embodiment, the nucleotide sequence encoding amino acids 1-550 of IDS comprises or consists of nucleotides 170-1822 of the human IDS cDNA as shown in FIG. 2B. However, the IDS nucleotide sequence may also be a codon-optimized sequence of the wild-type IDS nucleotide sequence. Hence, in other preferred embodiments, the nucleotide sequence encoding amino acids 26-550 or amino acids 1-550 of IDS comprises or consists of a codon-optimized sequence of nucleotides 245-1822 or nucleotides 170-1822, respectively, of the human IDS cDNA as shown in FIG. 2B. Codon optimization of nucleotide sequences encoding proteins is a well-known and commonly used technique in the art and a skilled person is well capable of preparing and selecting suitable codon optimized sequences. Suitable codon optimized IDS nucleotide sequences is used in Gleitz, H. F. et al., (2018, EMBO Mol Med 10. doi.10.15252/emmm.201708730).

Pompe Disease, also known as acid maltase deficiency or glycogen storage disease type II, is an autosomal recessive metabolic disorder. It is caused by an accumulation of glycogen in the lysosome. In Pompe Disease, the protein α-D-glucoside glucohydrolase or, in short, acid α-glucosidase (GAA, EC 3.2.1.20, also known as acid maltase), which is a lysosomal hydrolase, is defective. The protein is an enzyme that normally degrades the α-1,4 and α-1,6 linkages in glycogen, maltose and isomaltose and is required for the degradation of 1-3% of cellular glycogen. The deficiency of this enzyme results in the accumulation of structurally normal glycogen in lysosomes and cytoplasm in affected individuals. Excessive glycogen storage within lysosomes may interrupt normal functioning of other organelles and lead to cellular injury. A defective acid α-glucosidase protein, or reduced amount or activity of acid α-glucosidase protein, is the result of mutations (or variants) within the Glucosidase, alpha, acid (GAA) gene. The GAA gene is located on the long arm of chromosome 17 at 17q25.2-q25.3 (base pairs 80,101,556 to 80,119,879 in GRCh38.p10). Severe mutations that completely abrogate GAA enzyme activity cause a classic infantile disease course with hypertrophic cardiomyopathy, general skeletal muscle weakness, and respiratory failure and result in death within 1 years of life. Milder mutations leave partial GAA enzyme activity which results in a milder phenotype with onset varying from childhood to adult. In general, a higher residual enzyme activity in primary fibroblasts is associated with later onset of Pompe Disease. Some of the GAA mutations in Pompe Disease patients may lead to alternative splicing and thereby to absent or a reduced amount or activity of α-glucosidase protein. One of the most common mutations in Pompe Disease is the IVS1 mutation, c.-32-13T>G, a transversion (T to G) mutation which occurs among infants, children, juveniles and adults with this disorder (Huie M L, et al., 1994. Hum Mol Genet. 3(12):2231-6). In childhood and adult Pompe Disease, 90% of the patients in the Caucasian population are affected by the common c.32-13T>G (IVS1) variant that results in aberrant splicing of exon 2, such that exon 2 is partially or completely skipped. Absence of exon 2 from the mRNA results in absence of the normal AUG translation start site of the protein, which results in mRNA decay and failure to generate GAA protein.

The human GAA amino acid sequence encoded by the human GAA gene is shown in FIG. 3A. This sequence contains a signal peptide (amino acids 1-27) and a 110 kD preproprotein (amino acids 57-952) which is proteolytically further processed to generate multiple intermediate forms and the mature, active 76 kD and 70 kD forms of the GAA enzyme. The human GAA cDNA sequence, including the sequence encoding the signal peptide and the sequence encoding the preproprotein, is shown in FIG. 3B.

In some embodiments, the metabolic protein is acid alpha glucosidase (GAA). In preferred embodiments, the nucleic acid molecule of the invention comprises a nucleotide sequence encoding an amino acid sequence comprising a sequence having at least 90% sequence identity with amino acids 70-952 of human GAA. In further preferred embodiments, the nucleic acid molecule of the invention comprises a nucleotide sequence encoding an amino acid sequence comprising amino acids 28-952 of human GAA, or an amino acid sequence having at least 90% sequence identity with amino acids 28-952 of human GAA. In further preferred embodiments, said nucleotide sequence encodes an amino acid sequence having at least 90% sequence identity with amino acids 28-69 of human GAA and encodes amino acids 70-952 of human GAA. In further preferred embodiments, a nucleotide sequence encoding the GAA signal peptide is present. I.e. the nucleic acid molecule of the invention comprises a nucleotide sequence encoding an amino acid sequence comprising a sequence having at least 90% sequence identity with amino acids 1-985 or amino acids 1-27 and 70-952 of human GAA. Alternatively, another signal peptide is present, such as the IGFII signal peptide, see FIG. 1.

In some preferred embodiments, a nucleic acid molecule of the invention comprises a nucleotide sequence encoding amino acids 28-952 of human GAA, or an amino acid sequence having at least 95%, more preferably at least 96%, more preferably at least 97%, more preferably at least 98%, more preferably at least 99%, sequence identity with amino acids 28-952 of human GAA, or an amino acid sequence having at least 90% sequence identity with amino acids 28-952 of human GAA.

In some preferred embodiments, the variation in amino acid sequence is only in amino acids 28-69. I.e. in preferred embodiments, the nucleic acid molecule encodes an amino acid sequence having at least 90%, more preferably at least 95%, more preferably at least 96%, more preferably at least 97%, more preferably at least 98%, more preferably at least 99%, sequence identity with amino acids 28-69 of human GAA and encodes amino acids 70-952 of human GAA.

In one preferred embodiment, the nucleotide sequence encoding amino acids 70-952 of GAA comprises or consists of nucleotides 365-3013 of the human GAA cDNA as shown in FIG. 3B. In another preferred embodiment, the nucleotide sequence encoding amino acids 28-952 of GAA comprises or consists of nucleotides 239-3013 of the human GAA cDNA as shown in FIG. 3B. However, the GAA nucleotide sequence may also be a codon-optimized sequence of the wild-type GAA amino acid sequence. Hence, in other preferred embodiments, the nucleotide sequence encoding amino acids 70-952 or amino acids 28-952 of GAA comprises or consists of a codon-optimized sequence of nucleotides 365-3013 or nucleotides 239-3013, respectively, of the human GAA cDNA as shown in FIG. 3B. Codon optimization of nucleotide sequences encoding proteins is a well-known and commonly used technique in the art and a skilled person is well capable of preparing and selecting suitable codon optimized sequences. Suitable codon optimized GAA nucleotide sequences is described in Stok et al., 2020 (Molecular Therapy-Methods & Clinical Development 17:1014-1025).

Neuronal ceroid lipofuscinoses (NCLs) are a heterogeneous group of LSDs. CLN2 disease is caused by mutations in the tripeptidyl peptidase 1 (TPP1)/CLN2 gene, resulting in TPP1 enzyme deficiency. CLN2 most commonly presents with seizures and/or ataxia in the late-infantile period (ages 2-4), symptoms include language delay, progressive childhood dementia, motor and visual deterioration, and early death. Atypical phenotypes of CLN2 can occur and are characterized by later onset of the disease. Some atypical phenotypes are further characterized by longer life expectancies. Many mutations in TPP1 have been identified, but two variants, the c.509-1 G>C and c.622 C>T (p. (Arg208*)) mutations, occur in 60% of affected individuals and account for 50% of disease-associated alleles. The only current treatment available for CLN2 is cerliponase alfa (Brineura®).

The human TPP1 amino acid sequence encoded by the human TPP1/CLN2 gene is shown in FIG. 4. This sequence contains a signal peptide (amino acids 1-19). The human TPP1 cDNA sequence, including the sequence encoding the signal peptide is shown in FIG. 4B.

In some preferred embodiments, the metabolic protein is TPP1. In preferred embodiments, the nucleic acid molecule of the invention comprises a nucleotide sequence encoding an amino acid sequence comprising a sequence having at least 90% sequence identity with amino acids 20-563 of human TPP1. In further preferred embodiments, said nucleotide sequence encodes amino acids 20-563 of human TPP1. In further preferred embodiments, a nucleotide sequence encoding the TPP1 signal peptide is present. I.e. the nucleic acid molecule of the invention comprises a nucleotide sequence encoding an amino acid sequence comprising a sequence having at least 90% sequence identity with amino acids 1-563 of human TPP1. Alternatively, another signal peptide is present, such as the IGFII signal peptide, which is shown in FIG. 1.

In some preferred embodiments, a nucleic acid molecule of the invention comprises a nucleotide sequence encoding amino acids 20-563 of human TPP1, or an amino acid sequence having at least 95%, more preferably at least 96%, more preferably at least 97%, more preferably at least 98%, more preferably at least 99%, sequence identity with amino acids 20-563 of human TPP1, or an amino acid sequence comprising or consisting of amino acids 20-563 of human TPP1.

In some preferred embodiments, a nucleic acid molecule of the invention comprises a nucleotide sequence encoding amino acids 1-563 of human TPP1, or an amino acid sequence having at least 95%, more preferably at least 96%, more preferably at least 97%, more preferably at least 98%, more preferably at least 99%, sequence identity with amino acids 1-563 of human TPP1, or an amino acid sequence comprising or consisting of amino acids 1-563 of human TPP1.

In some preferred embodiments, the variation in amino acid sequence is only in amino acids 20-563. I.e. in some preferred embodiments, the nucleic acid molecule encodes an amino acid sequence having at least 90%, more preferably at least 95%, more preferably at least 96%, more preferably at least 97%, more preferably at least 98%, more preferably at least 99%, sequence identity with amino acids 20-563 of human TPP1 and encodes amino acids 1-19 of human TPP1.

In one preferred embodiment, the nucleotide sequence encoding amino acids 20-563 of TPP1 comprises or consists of nucleotides 80-1711 of the human TPP1 cDNA as shown in FIG. 4B. In another preferred embodiment, the nucleotide sequence encoding amino acids 1-563 of TPP1 comprises or consists of nucleotides 23-1711 of the human TPP1 cDNA as shown in FIG. 4B. However, the TPP1 nucleotide sequence may also be a codon-optimized sequence of the wild-type TPP1 amino acid sequence. Hence, in other preferred embodiments, the nucleotide sequence encoding amino acids 20-563 or amino acids 1-563 of TPP1 comprises or consists of a codon-optimized sequence of nucleotides 80-1711 or nucleotides 23-1711, respectively, of the human TPP1 cDNA as shown in FIG. 4B. Codon optimization of nucleotide sequences encoding proteins is a well-known and commonly used technique in the art and a skilled person is well capable of preparing and selecting suitable codon optimized sequences.

The nucleotide sequence encoding a metabolic protein or a part thereof or a sequence having at least 90% sequence identity to said metabolic protein or part thereof, the human IGFII gene sequence and the nucleotide sequence encoding at least one peptide that facilitates cellular uptake or transcytosis, preferably at least one peptide that facilitates passage over the BBB, are linked, meaning that expression of the nucleotide sequence encoding a metabolic protein or a part thereof or a sequence having at least 90% sequence identity to said metabolic protein or part thereof, the human IGFII gene sequence, and the nucleotide sequence encoding at least one peptide that facilitates cellular uptake or transcytosis, preferably at least one peptide that facilitates passage over the BBB, results in a proteinaceous compound, in particular a fusion protein, comprising the amino acids sequences encoded by the nucleotide sequence encoding a metabolic protein or a part thereof or a sequence having at least 90% sequence identity to said metabolic protein or part thereof, the human IGFII gene sequence, and the nucleotide sequence encoding at least one peptide that facilitates cellular uptake or transcytosis, preferably at least one peptide that facilitates passage over the BBB.

In preferred embodiments, the human IGFII gene sequence and the nucleotide sequence encoding at least one peptide that facilitates cellular uptake or transcytosis, preferably at least one peptide that facilitates passage over the BBB, are located 3′ of the nucleotide sequence encoding metabolic protein or a part thereof or a sequence having at least 90% sequence identity to said metabolic protein or part thereof. For instance, this is preferably the case if the metabolic protein is an IDS sequence, or part or variant thereof or a TPP1 sequence, or part of variant thereof.

In other preferred embodiments, the human IGFII gene sequence and the nucleotide sequence encoding at least one peptide that facilitates cellular uptake or transcytosis, preferably at least one peptide that facilitates passage over the BBB, are located 5′ of the nucleotide sequence encoding metabolic protein or a part thereof or a sequence having at least 90% sequence identity to said metabolic protein or part thereof. For instance, this is preferably the case if the metabolic protein is GAA, or part or variant thereof.

The nucleotide sequence encoding at least one peptide that facilitates cellular uptake or transcytosis, preferably at least one peptide that facilitates passage over the BBB, is inserted at a location between the nucleotides encoding of amino acids 28 and 42 of mature human IGFII of said IGFII gene sequence. It is hypothesized that this nucleotide sequence forms a loop within the IGFII sequence such that it is exposed at the outside of the IGFII sequence. It is hypothesized that insertion at this specific location provides for an excellent accessibility of the at least one peptide that facilitates cellular uptake or transcytosis, preferably at least one peptide that facilitates passage over the BBB. The accessibility enables binding of the at least one peptide to its receptor, in particular expressed on ECs that are part of the BBB. Furthermore, the at least one peptide is placed in a specific, fixed conformation relative to the IGFII peptide, as compared to placing the at least one peptide N- or C-terminal of the IGFII peptide. It is hypothesized that this is helpful for receptor binding and/or dimerization and to avoid steric hindrance between the different epitope tags. Finally, by inserting the at least one peptide at this location, insulin binding affinity is reduced without impairing brain targeting of the IGFII peptide and without optimizing a c-terminal or n-terminal tagging of a double or multi tag.

Insertion of the nucleotide sequence encoding the at least one peptide that facilitates cellular uptake or transcytosis is suitably combined with an IGFII sequence that has a mutation, preferably a deletion of one or more amino acids, within the nucleotide region encoding amino acids 29-41 of human mature IGFII. For instance, the nucleotide sequence can be inserted at the site of the deleted amino acids. In preferred embodiments, the nucleotide sequence is inserted at the site of a deletion of the nucleotides encoding at least amino acids 29-41, 30-41, 31-41, 32-41, 33-41, 34-41, 35-41, 36-41, 37-41, 29-40, 30-40, 31-40, 32-40, 33-40, 34-40, 35-40, 36-40, 37-40, 29-39, 30-39, 31-39, 32-39, 33-39, 34-39, 35-39, 36-39, 29-38, 30-38, 31-38, 32-38, 33-38, 34-38, 35-38, 36-38, 29-37, 30-37, 31-37, 32-37, 33-37, 34-37, 35-37, 29-36, 30-36, 31-36, 32-36, 33-36, 34-36, 29-35, 30-35, 31-35, 32-35, 33-35, 29-34, 30-34, 31-34, 30-34, 29-33, 30-33, 31-33, 29-32 or 30-32 of human mature IGFII as shown in FIG. 1. In a particularly preferred embodiment, the nucleotides encoding amino acids 29-41 of mature human IGFII are deleted and the nucleotide sequence is inserted between the nucleotides encoding amino acids 28 and 42 of mature human IGFII. It is further preferred that such IGFII sequence comprises or consists of nucleotides encoding amino acids 1 and 8-28 and 42-67 of human mature IGFII, as shown in FIG. 1. Optionally nucleotides encoding one or more amino acids may be present between the IGFII sequence and the nucleotide sequence encoding at least one peptide that facilitates cellular uptake or transcytosis, at one or both sides of the insertion. I.e. nucleotides encoding one or more amino acids that flank the inserted nucleotide sequence encoding at least one peptide that facilitates cellular uptake or transcytosis. In a preferred embodiments, nucleotides encoding at least a cysteine are present at each side of the insertion. It is believed that this allows the formation of disulfide bonds between the cysteine residues flanking the insertion in the encoded fusion protein, such that the amino acids of the IGFII sequence that are closest to the inserted nucleotide sequence, such as amino acids 28 and 42 of mature human IGFII in case of a deletion amino acids 29-41, are in proximity. In addition to the nucleotides encoding a cysteine residue, addition linking sequence may be present at one or both sides of the insertion. In the examples herein, constructs 5-14 are examples of constructs wherein the nucleotide sequence encoding at least one peptide that facilitates cellular uptake or transcytosis is inserted between the nucleotides encoding of amino acids 28 and 42 of said IGFII gene sequence. Constructs 6, 8 and 9 contain two linking sequences flanking the inserted nucleotide sequence. Constructs 5, 7, and 10-14 contain two linking sequence and cysteine encoding nucleotides flanking the inserted nucleotide sequence.

Hence, in a preferred embodiment, in a nucleic acid molecule of the invention, the IGFII gene sequence comprises or consists of nucleotides encoding amino acids 1 and 8-28 and 42-67 of human mature IGFII, the nucleotide sequence encoding at least one peptide that facilitates cellular uptake or transcytosis, preferably at least one peptide that facilitates passage over the BBB, is inserted between the nucleotides encoding of amino acids 28 and 42 of said IGFII gene sequence and nucleotide sequences encoding a cysteine and/or a linking sequence are present between the nucleotides encoding amino acid 28 of the mature human IGFII sequence and the nucleotide sequence encoding at least one peptide that facilitates cellular uptake or transcytosis, preferably at least one peptide that facilitates passage over the BBB, and between the nucleotide sequence encoding at least one peptide that facilitates cellular uptake or transcytosis, preferably at least one peptide that facilitates passage over the BBB, and the nucleotides encoding amino acid 42 of mature human IGFII. If both cysteine encoding nucleotides and linking sequences are present, it is preferred that the linking sequence is adjacent to the nucleotide sequence encoding at least one peptide that facilitates cellular uptake or transcytosis, preferably at least one peptide that facilitates passage over the BBB, at both sides thereof.

As indicated herein above, one or more linking sequences may be present in a nucleic acid molecule of the invention. In preferred embodiments, the nucleic acid molecule comprises linking sequences flanking the nucleotide sequence encoding at least one peptide that facilitates cellular uptake or transcytosis if this is inserted between nucleotides encoding amino acids 28 and 42 of human mature IGFII.

In preferred embodiments, the linking sequence is a nucleotide sequence that encodes an amino acid sequence of 2-30 amino acids, preferably of 2-25 amino acids, more preferably of 2-22 amino acids, such as 4, 6, 10, 14, 15, 20 or 21 amino acids. In preferred embodiments, the linking sequence encode an amino acid sequence having a high percentage of G and S residue, such as at least 50%, or at least 60% or at least 70%. Suitable, but non-limiting linking sequences are nucleotide sequence that encodes amino acid sequences LGGGGSGGGGSGGGGSGGGGS, SGGGG, SGGGGSG, GAPLGGGGSGGGGS, SGGSGGGGSGGGGSG, SGGS, GGSGGSGGSG.

The linking sequences flanking the nucleotide sequence encoding at least one peptide that facilitates cellular uptake or transcytosis and that is inserted between amino acids 28 and 42 of the IGFII gene sequence may be the same or different. As detailed herein above, if linking sequences are present flanking the nucleotide sequence encoding at least one peptide that facilitates cellular uptake or transcytosis, it is preferred that nucleotides encoding a cysteine residue are further present attached to both linking sequences, adjacent to amino acids 28 and 42 of the IGFII sequence.

A nucleic acid molecule of the invention preferably comprises a signal peptide. The signal peptide can be any signal peptide that is suitable for secretion of a fusion protein encoded by a nucleic acid molecule of the invention, or fusion protein of the invention. Suitable signal peptides are known in the art. In preferred embodiments, the signal peptide is a signal peptide of a metabolic protein, preferably lysosomal enzyme, preferably lysosomal hydrolase. In further preferred embodiments, the signal peptide is a signal peptide of the metabolic protein, preferably lysosomal enzyme, preferably lysosomal hydrolase that is present in the nucleic acid molecule of the invention or in the fusion protein of the invention. E.g. if the metabolic protein is IDS, the signal peptides is preferably the IDS signal peptide. The signal peptide is preferably present 5′ of the nucleotide sequence encoding a metabolic protein or a part thereof or a sequence having at least 90% sequence identity to said metabolic protein or part thereof, the human insulin-like growth factor II (IGFII) gene sequence, and the nucleotide sequence encoding at least one peptide that facilitates cellular uptake or transcytosis. The signal peptide is preferably present at the N-terminus of the fusion protein encoded by the nucleic acid molecule or of the invention.

A nucleic acid molecule of the invention may further comprise one or more regulatory sequences to direct expression of one or more of the nucleotide sequences present on the nucleic acid molecule. Examples include a promoter, an enhancer, a transcription termination signal, and a polyadenylation sequence.

In preferred embodiments a nucleic acid molecule of the invention comprises a promoter operably linked to the nucleotide sequence encoding a metabolic protein or a part thereof or a sequence having at least 90% sequence identity to said metabolic protein or part thereof, human IGFII gene sequence, and nucleotide sequence encoding at least one peptide that facilitates cellular uptake or transcytosis, preferably at least one peptide that facilitates passage over the BBB. A used herein “operably linked” means that a nucleotide sequence is functionally associated with one or more other nucleotide sequences. For instance, the promoter nucleotide sequence is functionally associated with the nucleotide sequence encoding a metabolic protein or a part thereof or a sequence having at least 90% sequence identity to said metabolic protein or part thereof, human IGFII gene sequence, and nucleotide sequence encoding at least one peptide that facilitates cellular uptake or transcytosis, such that the promoter sequence influences or directs the expression of the other nucleotide sequences. Various promoters, including inducible promoters, may be used to direct expression of nucleotide sequences included in a nucleic acid molecule of the invention, including viral promoter. Both cell type specific an ubiquitous promoters can be used. Suitable promoters include a SV40 promoter, a Rous Sarcoma Virus (RSV) promoter, a cytomegalovirus (CMV) promoter, a CD11b promoter and an MND promoter or derivatives of any of these promoters.

In the case of LSDs accumulation and symptoms in several organs need to be corrected (including lungs, heart, skeletal and smooth muscle, joints, connective tissue, peripheral nervous system and brain) and the peripheral nervous system (see below). Therefore, in preferred embodiments the promoter is a ubiquitous promoter rather than a cell type specific promoter. In preferred embodiments the promoter is an MND promoter or derivatives thereof, such as the MND promoter with a 174 bp deletion at the 5′ end. MND promoter is described in Robbins et al (Proc. Natl. Acad. Sci. USA 1994, Vol. 95, pp. 10182-10187) and Astrakhan et al (Blood. 2012; 119(19): 4395-4407), which are incorporated herein by reference. In preferred embodiments, the metabolic protein is IDS and the nucleic acid molecule of the invention comprises a ubiquitous promoter.

Another strategy is to use a myeloid promoter, i.e. a promoter that is specific for cells from the myeloid lineage, such as the lysM, csf1r, CD11c, CD68, macrophage SRA, and CD11b promoters, since the secreted enzyme is preferably expressed from HSCs, which are in the myeloid lineage. Hence, in other preferred embodiments, the promoter is a myeloid promoter, such as the lysM, csf1r, CD11c, CD68, macrophage SRA, and CD11b promoters, preferably the CD11b promoter.

Expression of nucleic acid sequences present on a nucleic acid molecule of the invention results in a fusion protein comprising a metabolic protein or a part thereof or a sequence having at least 90% sequence identity to said metabolic protein or part thereof fused to a human insulin-like growth factor II (IGFII) sequence and at least one peptide that facilitates cellular uptake or transcytosis.

Also provided is therefore a fusion protein encoded by the nucleic acid molecule according to the invention.

Further provided is a fusion protein comprising a metabolic protein or a part thereof or a sequence having at least 90% sequence identity to said metabolic protein or part thereof fused to a human insulin-like growth factor II (IGFII) sequence and at least one peptide that facilitates cellular uptake or transcytosis, preferably at least one peptide that facilitates passage over the BBB.

In preferred embodiments, the human IGFII sequence and the at least one peptide that facilitates cellular uptake or transcytosis, preferably at least one peptide that facilitates passage over the BBB, are located at the same side of the metabolic protein or a part thereof or sequence having at least 90% sequence identity to said metabolic protein or part thereof in a fusion protein of the invention.

In preferred embodiments of a fusion protein of the invention, said metabolic protein or part thereof or sequence having at least 90% sequence identity to said metabolic protein or part thereof, human IGFII sequence and/or said at least one peptide that facilitates cellular uptake or transcytosis, preferably at least one peptide that facilitates passage over the BBB, are separated by one or more linking sequences and/or cysteine residues. Such linking sequence, cysteine residues and the location of nucleotide sequences encoding these in the nucleic acid molecule of the invention are discussed herein above. The same disclosure and embodiments are applicable to a fusion protein of the invention.

In further preferred embodiments, the human IGFII amino acid sequence in a fusion protein of the invention is an amino acid sequence encoded by the IGFII gene sequence as defined herein above.

In preferred embodiments, the IGFII amino acid sequence comprises amino acids 1 and 8-67 of human IGFII, as shown in FIG. 1.

In further embodiments, the IGFII amino acid sequence comprises the IGFII signal peptide and amino acids 1-67 of IGFII, as shown in FIG. 1.

In further preferred embodiments, the IGFII amino acid sequence comprises comprising a mutation within amino acids 29-41 of the IGFII sequence, preferably a deletion within amino acids 29-41 or 30-40 of IGFII.

In further preferred embodiments, the IGFII amino acid sequence comprises the IGFII signal peptide and human mature IGFII comprising a deletion of amino acids 29-41, 30-41, 31-41, 32-41, 33-41, 34-41, 35-41, 36-41, 37-41, 29-40, 30-40, 31-40, 32-40, 33-40, 34-40, 35-40, 36-40, 37-40, 29-39, 30-39, 31-39, 32-39, 33-39, 34-39, 35-39, 36-39, 29-38, 30-38, 31-38, 32-38, 33-38, 34-38, 35-38, 36-38, 29-37, 30-37, 31-37, 32-37, 33-37, 34-37, 35-37, 29-36, 30-36, 31-36, 32-36, 33-36, 34-36, 29-35, 30-35, 31-35, 32-35, 33-35, 29-34, 30-34, 31-34, 30-34, 29-33, 30-33, 31-33, 29-32 or 30-32 of human IGFII as shown in FIG. 1.

In particularly preferred embodiments, the IGFII amino acid sequence comprises a deletion of amino acids 29-41 or 30-40 of IGFII. Hence, in preferred embodiments, the IGFII amino acid sequence comprises amino acids 1, 8-28 and 42-67 or amino acids 1, 8-29 and 41-67 of mature IGFII, as shown in FIG. 1. Further suitable IGFII mutants with reduced insulin receptor binding are described in WO 2009/137721, which is incorporated herein by reference.

In preferred embodiments, the at least one peptide that facilitates cellular uptake or transcytosis, preferably at least one peptide that facilitates passage over the BBB, is inserted between amino acids 28-42 of the human IGFII sequence in a fusion protein of the invention. In particular the at least one peptide is inserted at the location of the mutation within amino acids 29-41 or 30-40 of the IGFII sequence. In particular, the at least one peptide is located at the location of the deleted amino acids in the IGFII sequence. I.e. the IGFII amino acid sequence comprises a deletion of amino acids 29-41 or 30-40 of IGFII and the at least one peptide that facilitates cellular uptake or transcytosis, preferably at least one peptide that facilitates passage over the BBB, is located at the site of the mutated amino acids. In preferred embodiments, the IGFII amino acid sequence comprises amino acids 1, 8-28 and 42-67 or amino acids 1, 8-29 and 41-67 of mature IGFII, as shown in FIG. 1, and the at least one peptide that facilitates cellular uptake or transcytosis, preferably at least one peptide that facilitates passage over the BBB, is located between amino acids 28 and 42 or 29 and 41, respectively, of the IGFII sequence. In preferred embodiments, a cysteine is present at each side of the insertion, i.e. of the at least one peptide that facilitates cellular uptake or transcytosis, preferably at least one peptide that facilitates passage over the BBB. In addition to the cysteine residues, additional linking sequences may be present at one or both sides of the insertion. If both cysteines and linking sequences are present, it is preferred that the linking sequence is adjacent to the at least one peptide that facilitates cellular uptake or transcytosis, preferably at least one peptide that facilitates passage over the BBB, at both sides thereof. I.e. the cysteines are preferably located adjacent to part of the sequence of the IGFII sequence, e.g. adjacent to amino acids 28 if amino acids 29-41 are deleted and 41 or adjacent to amino acids 29 and 41 if amino acids 30-40 are deleted.

The fusion protein preferably comprises a linking sequence as defined herein between the IGFII amino acid sequence and the GAA amino acid sequence. The linking sequence preferably consists of 2-10 amino acids, more preferably of 2-5 amino acids, such as 2, 3 or 4 amino acids. In preferred embodiments, the linking sequence is an amino acid sequence of 3 amino acids. A preferred, but non-limiting linking sequence is the amino acid sequence GAP.

Also provided is a nucleic acid sequence encoding a fusion peptide according to the invention.

After transplantation of HSC transfected or transduced with a nucleic acid molecule of the invention the cells express the fusion protein in vivo. This fusion protein contains an IGFII-tagged GAA preproprotein, contrary to known IGFII-tagged GAA fusion proteins expressed after HSC transfection or transduction and transplantation.

In preferred embodiments, a nucleic acid molecule of the invention is or is part of a vector. In preferred embodiments, the nucleic acid molecule of the invention is a gene therapy vector. Also provided by the invention is therefore a vector, preferably a gene therapy vector comprising a nucleic acid molecule of the invention.

The gene therapy vector is preferably a vector that is suitable for ex vivo transfection or transduction of haematopoietic stem cells.

The vector can be a viral or non-viral vector. Non-limiting examples of suitable expression vectors include retroviral, adenoviral, adeno-associated and herpes simplex viral vectors, non-viral vectors and engineered vectors.

The vector, in particular gene therapy vector, preferably is a viral vector. Said viral vector preferably is a recombinant adeno-associated viral vector, a herpes simplex virus-based vector, or a lentivirus-based vector such as a human immunodeficiency virus-based vector. Said viral vector further preferably is a retroviral-based vector such as a lentivirus-based vector such as a human immunodeficiency virus-based vector, or a gamma-retrovirus-based vector. Retroviruses can be packaged in a suitable complementing cell that provides Group Antigens polyprotein (Gag)-Polymerase (Pol) and/or Envelop (Env) proteins. Suitable packaging cells are human embryonic kidney derived 293T or 293 cells, Phoenix cells (Swift et al., 2001. Curr Protoc Immunol, Chapter 10: Unit 10 17C), PG13 cells (Loew et al., 2010. Gene Therapy 17:272-280) and Flp293A cells (Schucht et al., 2006. Mol Ther 14:285-92). Methods for the generation of such non-viral expression vectors are well known in the art.

Non-viral expression vectors include nude DNA, and nucleic acids packaged into synthetic or engineered compositions such as liposomes, polymers, nanoparticles and molecular conjugates. Methods for the generation of such non-viral expression vectors are well known in the art.

As an alternative, a nucleic acid molecule used in accordance with the invention may be provided to a subject by gene editing technology, including CRISPR/Cas, zinc-finger nucleases, and transcription activator-like effector nucleases-TALEN, in order to insert the receptor transgenes into specific genomic loci with or without an exogenous promoter. Preferred genomic loci include the AAVS1 locus and the PD-1 locus, as is known to a skilled person.

In particularly preferred embodiments, the nucleic acid molecule or vector of the invention is a gene delivery vehicle of lentiviral origin. In preferred embodiments the vector, in particular gene therapy vector is a lentiviral vector. Typically, the terms “gene delivery vehicle of lentiviral origin” and “lentiviral vector” refer to a gene delivery vehicle of any lentiviral origin. Gene delivery vehicles of lentiviral origin are all vehicles comprising genetic material and/or proteinaceous material derived from lentiviruses. Typically the most important features of such vehicles are the integration of their genetic material into the genome of a target cell and their capability to transduce stem cells. These elements are deemed essential in a functional manner, meaning that the sequences need not be identical to lentiviral sequences as long as the essential functions are present. The methods of the invention are however especially suitable for recombinant lentiviral particles, which have most if not all of the replication and reproduction features of a lentivirus, typically in combination with a producer cell having some complementing elements. Normally the lentiviral particles making up the gene delivery vehicle are replication defective on their own. In a preferred embodiment, the used gene delivery vehicle of lentiviral origin is a gene delivery vehicle of HIV lentiviral origin and even more preferred are HIV-1 derived self-inactivating lentiviral vectors.

The invention also provides a viral particle comprising a nucleic acid molecule according to the invention, preferably a lentiviral particle.

Methods and means (such as producer cells) for producing the desired lentiviral particles are well known in the art. Examples of preferred producer cells are 293T cells that are co-transfected with VSV-G (Vesicular stomatitis virus-G-protein envelope). Other pseudotypes used in this setting are RD114 (Feline-immunodeficiency virus).

The invention also provides uses of the nucleic acid molecules, vectors, fusion proteins, compositions and cell populations, in particular HSC populations, particularly in the treatment of a metabolic disorder.

In one aspect, the invention therefore also provides a method for the treatment of a metabolic disorder comprising administering a nucleic acid molecule, fusion protein or cell population, in particular HSC, according to the invention to an individual in need thereof.

Also provided is a nucleic acid molecule, fusion protein or cell population, in particular HSC, according to the invention for use in a method for the treatment of a metabolic disorder.

Also provided is the use of a nucleic acid molecule, fusion protein or cell population, in particular HSC, according to the invention in the preparation of a medicament for the treatment of a metabolic disorder.

As used herein, “metabolic disorder”, also mentioned metabolic disease, refers to a disorder or disease wherein normal cellular metabolism is disturbed. The metabolic disorder is in particular a disorder or disease in which expression and/or functional activity of a metabolic enzyme is impaired or absent. Metabolic enzymes are enzymes involved in cellular metabolism. In preferred embodiments, the metabolic disorder is a lysosomal disorder, more preferably a lysosomal storage disorder. As used herein, “lysosomal disorder” or “lysosomal disease” refers to a metabolic disorder resulting from a defect in lysosomal function. A “lysosomal storage disorder” (LSD) refers to a metabolic disorder characterized by lysosomal dysfunction and accumulation of non-degraded substrate in the lysosomes. Non-limiting examples of LSD are Fabry disease, Farber lipogranulomatosis, Gaucher disease, GM1 gangliosidosis, Tay-Sachs disease, GM2 gangliosidosis, Krabbe disease, metachromatic leukodystrophy, Niemann-Pick A/B, Wolman disease, cholesterol ester storage disease, PMS I or Hurler syndrome, PMSII or Hunter syndrome, PMS IIIA, PMS IIIB, PMS IIIC, PMS IIID, PMS IVA, PMS IVB, PMS VI, PMS VII, PMS IX, multiple sulfatase deficiency, mucolipidosis II α/β, I-cell disease, pseudo-Hurler polydystrophy, mucolipidosis III γ, α-mannosidosis, β-mannosidosis, fucosidosis, aspartylglucosaminuria, Schindler disease, sialidosis type I or II, galactosialidosis, Hermanski-Pudlak disease type 1, 2, 3, 4, 5, 6, 7, 8 or 9, Griscelli syndrome 1 or 2, Chediak-Higashi disease, Pompe disease, Neuronal ceroid lipofuscinoses (CLN) 1, 2, 3, 4, 5, 6, 7, 8, 10, 11, 12, 13 or 14 . . . . In one preferred embodiment, the LSD treated in accordance with the invention is Hunter syndrome or PMSII, and the metabolic protein is IDS or a part thereof or a sequence having at least 90%, 95%, 96%, 97%, 98% or 99% sequence identity with IDS or part thereof. In another preferred embodiment, the LSD treated in accordance with the invention is Pompe disease, and the metabolic protein is GAA or a part thereof or a sequence having at least 90%, 95%, 96%, 97%, 98% or 99% sequence identity with GAA or part thereof. In another preferred embodiment, the LSD treated in accordance with the invention is CLN2, and the metabolic protein is TPPI or a part thereof or a sequence having at least 90%, 95%, 96%, 97%, 98% or 99% sequence identity with TPPI or part thereof.

In preferred embodiments of the invention, a nucleic acid molecule or vector of the invention is for use in transfecting or transducing cells, in particular hematopoietic stem cells (HSC), preferably ex vivo transfecting or transducing cells, in particular HSC.

In one aspect, the invention therefore provides a method for transfecting or transducing HSC with a nucleic acid molecule or vector, preferably a gene delivery vehicle of lentiviral origin, according to the invention comprising contacting HSC with said nucleic acid molecule or vector, preferably gene delivery vehicle. In preferred embodiments, said HSC are human, more preferably isolated from an individual suffering from a LSD, in particular Hunter syndrome, Pompe disease or CLN2. As the skilled person will understand, the specific LSD is dependent on the specific metabolic protein or part thereof that is encoded by a nucleotide sequence present on the nucleic acid molecule or vector.

As used herein, the term “transfection” refers to the transfer of genetic material from a non-viral particle to a hematopoietic stem cell. As used herein, the term “transduction” refers to the transfer of genetic material from a viral particle to a hematopoietic stem cell.

HSC are stem cells and the early precursor cells which give rise to all the blood cell types that include both the myeloid (monocytes and macrophages, neutrophils, basophils, eosinophils, erythrocytes, megakaryocytes/platelets and some dendritic cells) and lymphoid lineages (T-cells, B-cells, NK-cells, some dendritic cells). The hematopoietic tissue has cells with long term and short term regeneration capacities and committed multipotent, oligopotent and unipotent progenitors. HSC can be obtained from different sources and are, for example, found in the bone marrow of humans, which includes femurs, hip, ribs, sternum, and other bones. Cells can be obtained directly by removal from the hip using a needle and syringe, or from the blood following pre-treatment with cytokines, such as G-CSF (granulocyte colony stimulating factor), that induces cells to be released from the bone marrow compartment into the blood (mobilized peripheral blood).

In preferred embodiments, autologous HSC are transfected or transduced with a nucleic acid molecule or vector of the invention, i.e. HSC obtained from the patient. In such embodiments, HSC are preferably isolated from an individual suffering from a LSD to be treated, in particular Hunter syndrome, Pompe disease or CLN2, transfected or transduced with a nucleic acid molecule or vector of the invention ex vivo. After transfection or transducing, the transfected or transduced HSC are preferably returned to the individual. As the skilled person will understand, the specific LSD is dependent on the specific metabolic protein or part thereof that is encoded by a nucleotide sequence present on the nucleic acid molecule or vector.

The invention also provides compositions obtainable by the methods of the invention. Thus provided are compositions comprising HSC transfected or transduced with a nucleic acid molecule or vector, preferably a gene delivery vehicle of lentiviral origin, of the invention.

The invention further provides a composition comprising a viral particle, preferably, lentiviral particles, provided with a nucleic acid molecule or vector of the invention. Preferable, said lentiviral particles are gene delivery vehicles and capable of transducing HSC and/or progenitor cells.

The invention further provides a cell population, preferably a hematopoietic stem cells (HSC) population, provided with a nucleic acid molecule, vector or viral particle according to the invention. In preferred embodiments, the cell population or HSC is/are transfected or transduced with a nucleic acid molecule, vector or viral particle according to the invention. The cell population or HSC is/are further preferably capable of expression a fusion protein according to the invention, in particular a fusion protein comprising a metabolic protein or a part thereof or a sequence having at least 90% sequence identity to said metabolic protein or part thereof fused to a human insulin-like growth factor II (IGFII) sequence and at least one peptide that facilitates cellular uptake or transcytosis, preferably at least one peptide that facilitates passage over the BBB. In preferred embodiments, the cell population, preferably HSC population, expresses a fusion protein comprising a metabolic protein or a part thereof or a sequence having at least 90% sequence identity to said metabolic protein or part thereof fused to a human insulin-like growth factor II (IGFII) sequence and at least one peptide that facilitates cellular uptake or transcytosis of the invention.

A composition, cell population or HSC according to, or used in accordance with, the invention can be administered to an individual by a variety of routes, preferably by parenteral administration. Parenteral administration can include, for example, intraarticular, intramuscular, intravenous, intraventricular, intraarterial or intrathecal administration. In preferred embodiments, a composition or HSC of the invention may be administered to an individual via infusion or injection, in particular in hospital via infusion or via injection by a healthcare professional.

Compositions of the invention preferably comprise at least one pharmaceutically acceptable carrier, diluent and/or excipient. By “pharmaceutically acceptable” it is meant that the carrier, diluent or excipient must be compatible with the other ingredients of the composition and preferably not deleterious, e.g. toxic, to the recipient thereof. In general, any pharmaceutically suitable additive which does not interfere with the function of the active compounds can be used. A composition including the at least one pharmaceutically acceptable carrier, diluent and/or excipient according to the invention is preferably suitable for human use.

A composition, cell population or HSC for intravenous administration may for example be aqueous or non-aqueous sterile solutions of the HSC of the invention, for instance solutions in sterile isotonic aqueous buffer. Where necessary, the intravenous compositions may include for instance one or more buffers, solubilizing agents, stabilizing agents and/or a local anesthetic to ease the pain at the site of the injection.

Typically the use of a composition comprising lentiviral particles involves the transduction of HSC such as bone marrow cells, umbilical cord blood cells or mobilized peripheral blood stem cells. Such transduced cells are preferably prepared ex vivo and are also part of the present invention. Thus the invention provides a composition for the treatment of a metabolic disorder, preferably LSD, more preferably Hunter syndrome, Pompe disease or CNL2, comprising a plurality HSC transduced with a composition of lentiviral vector or particles according to the invention.

In yet another embodiment, the invention provides the use of a composition as described above in the preparation of a medicament for the treatment of a metabolic disorder, preferably LSD, more preferably Hunter syndrome, Pompe disease or CNL2.

In one aspect, the invention provides a method for treating a metabolic disorder, preferably LSD, more preferably Hunter syndrome, Pompe disease or CNL2, comprising administering the cell population comprising a cell population, in particular HSC, according to the invention to an individual in need thereof.

Also provided is a method for treating a metabolic disorder, preferably LSD, more preferably Hunter syndrome, Pompe disease or CNL2, comprising administering to an individual in need thereof a cell population, in particular HSC, that are transfected or transduced ex vivo with a nucleic acid molecule, a vector or viral particles, preferably a lentiviral vector or lentiviral particles, according to the invention. Also provided is a cell population, preferably HSC, transfected or transduced with a nucleic acid molecule or vector according to the invention for use in a method for the treatment of a metabolic disorder, preferably LSD, more preferably Hunter syndrome, Pompe disease or CNL2, in an individual. In preferred embodiments, the cells, in particular HSC, have been isolated from the individual and have been transfected or transduced ex vivo with a nucleic acid molecule or vector of the invention. I.e. in preferred embodiments individuals are treated with autologous transfected or transduced HSC. Hence, in preferred embodiments, a method of treatment of the invention comprises removing HSC from the individual, providing said HSC with a nucleic acid molecule, vector or viral particle according to the invention, preferably lentiviral vector or lentiviral particles, and administering said HSC provided with said nucleic acid molecule, vector or viral particle, preferably lentiviral vector or lentiviral particles, to said individual. In preferred embodiments, a method or use comprises transfecting or transducing said HSC with the nucleic acid molecule, vector or viral particle according to the invention, preferably a lentiviral vector or lentiviral particles, and administering said HSC transfected or transduced with said nucleic acid molecule, vector or viral particle, preferably lentiviral vector or lentiviral particles, to said individual. However, allogenic HSC may also be used in the methods and uses of the invention.

In preferred embodiments, a method or use of the invention comprises providing the individual with myeloablative treatment prior to administering treatment, in particular transfected or transduced HSC, according to the invention. Myeloablative treatment is also referred to in the art as myeloablative conditioning or myeloablative preconditioning. The term refers to treatment, such as chemotherapy or irradiation that eliminate hematopoietic cells of the individual treated. Preferably, most (e.g. more than 80% of) hematopoietic cells are eliminated. In addition, reduced preconditioning regimens may be applied. Myeloablative treatment is preferably performed by chemotherapy. Myeloablative treatment of human individuals is well known in the art and suitable chemotherapeutics are well known and can be selected by a person skilled in the art. Such agents are for instance used for allogeneic stem cell transplantation. Suitable, but non-limiting examples of such myeloablative agents are busulfan, melphalan, treosulfan fludarabine, cyclophosphamide, and combinations thereof. In preferred embodiments, the myeloablative treatment is with an agent selected from the group consisting of busulfan, treosulfan, fludarabine and combinations thereof.

In further preferred embodiments, the treatment, in particular HSC transplantation, in accordance with the present invention is combined with enzyme replacement therapy (ERT). The enzyme of the ERT is preferably the same as the metabolic protein or part thereof that is present in a fusion protein or encoded by a nucleotide sequence present in the nucleic acid molecule of the invention. E.g. if the fusion protein comprises IDS or a part thereof or the nucleic acid molecule comprises a nucleotide sequence encoding IDS or a part thereof, the enzyme used in ERT is IDS or part or variant thereof optionally in combination with or fused to a chaperone, an agent or a peptide that facilitates passage across the blood brain barrier. If the fusion protein comprises GAA or a part thereof or the nucleic acid molecule comprises a nucleotide sequence encoding GAA or a part thereof, the enzyme used in ERT is GAA or part or variant thereof optionally in combination with or fused to a chaperone, an agent or a peptide that facilitates passage across the blood brain barrier. A “part” or “variant” is a part or variant of the relevant enzyme that has the same kind of enzymatic activity.

In some embodiments, the individual is treated with ERT prior to treatment, in particular HSC transplantation, in accordance with the invention. In such cases, ERT for instance serves to improve the condition of the patient prior to treatment, in particular HSC transplantation, in accordance with the invention. In some preferred embodiment, the individual is treated with an immune suppressive agent prior to or concomitant with ERT. Immune suppressive agents are well known in the art and suitable immune suppressive agent are well known and can be selected by a person skilled in the art. As used herein an immune suppressive agent refers to an agent is capable of suppressing immune reaction in an individual. In preferred embodiments, B cells are suppressed. In preferred embodiments, the immune suppressive agent is selected from the group consisting of a B cell depletion agent, such as an anti-CD20 antibody, a T cell depletion agent, such as an anti-CD3 antibody (such as OKT3, muronomab) or an anti T cell receptor antibody (such as Muromonab-CD3), an anti-IL-2 receptor antibody (such as basiliximab and daclizumab), azathioprine, a calcineurin inhibitor, a corticosteroid, cyclosporine, methotrexate, IVIG, mercaptopurine, mycophenolate mofetil, and combinations thereof. In further preferred embodiments the immune suppressive agent is a B cell depletory agent, such as an anti-CD20 antibody, in particular rituximab. As used herein, a B cell depletion agent is an agent that reduces the amount of B cells in the individual that is treated with the agent. Treatment with an immune suppressing agent may reduce or prevent the formation of antibodies specific for the relevant enzyme, which is associated with ERT. Therefore such treatment optimizes combination treatment with ERT and treatment, in particular HSC transplantation, in accordance with the invention.

In some embodiments, the individual is treated with ERT during and/or subsequent to treatment, such as HSC transplantation, in accordance with the invention. In some embodiments, the individual is treated with ERT both prior to and during and/or subsequent to treatment, such as HSC transplantation, in accordance with the invention. In some embodiments, the individual is not treated with ERT but only with treatment, in particular HSC transplantation, in accordance with the invention.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: Human IGFII amino acid sequence (based on P01344; UniProt).

FIG. 2: IDS sequences. A. Human IDS amino acid sequence (NP_000193.1). B. Human IDS mRNA sequence (nucleotides 170-1822 of NM_000202.8).

FIG. 3: GAA sequences. A. Human GAA amino acid sequence (NP_000143.2; GenPept). B. Human GAA mRNA sequence (NM_001079803.3; GenBank).

FIG. 4: TPP1 sequences. A. Human TPP1 amino acid sequence (NP_000382.3). B. Human TPP1 mRNA sequence (nucleotides 23-1711 of NM_000391.4).

FIG. 5: Lentiviral vectors coding for IDS and tagged variants of IDS.
IDSco: codon optimized nucleotide sequence coding for IDS aa 26-550
IDS SP: codon optimized nucleotide sequence coding for IDS aa 1-25 corresponding to the IDS signal peptide
IGF2: codon optimized nucleotide sequence coding for the human mature IGF2 aa 1, 8-67. aa 1 is intended as the 1st aa of human mature IGF2 after the signal peptide aa 2-7 are deleted.
ApoE2x2: codon optimized nucleotide sequence coding for aa (LRKLRKRLL)x2. Sequence LRKLRKRLL is aa 141-149 of human apolipoprotein E.
ApoE2: codon optimized nucleotide sequence coding for aa LRKLRKRLL corresponding to aa 141-149 of the human apolipoprotein E.
RAP12x2: codon optimized nucleotide sequence coding for aa EAKIEKHNHYQKGEAKIEKHNHYQK, where sequence EAKIEKHNHYQK is aa 251-262 of the human Receptor Associated Protein (RAP, also called alpha 2-macroglobulin receptor-associated protein) domain 3.
RAP12x1: codon optimized nucleotide sequence coding for aa EAKIEKHNHYQK, aa 251-262 of the human Receptor Associated Protein (RAP, also called alpha 2-macroglobulin receptor-associated protein) domain 3.
ApoB: codon optimized nucleotide sequence coding for aa SSVIDALQYKLEGTTRLTRKRGLKLATALSLSNKFVEGS corresponding to aa 3371-3409 of the human apolipoprotein B.
IGF2a: sequence ALCGGELVDTLQFVCGDRGFYF corresponding to aa 1, 8-28 of the mature human IGF2. aa 1 is intended as the 1st aa of mature human IGF2 after the signal peptide.
IGF2b: sequence SG corresponding to aa 29 and 41 of the mature human IGF2. aa 1 is intended as the 1st aa of mature human IGF2 after the signal peptide.
IGF2c: sequence IVEECCFRSCDLALLETYCATPAKSE corresponding to aa 42-67 of the mature human IGF2. aa 1 is intended as the 1st aa of mature human IGF2 after the signal peptide
Angiopep2: codon optimized nucleotide sequence coding for aa TFFYGGSRGKRNNFKTEEY
Leptin-30: codon optimized nucleotide sequence coding for aa YQQILTSMPSRNVIQISNDLENLRDLLHVL
SP: signal peptide
co: codon optimized
L: linker sequence aa LGGGGSGGGGSGGGGSGGGGS
L1: linker sequence SGGGG
L2: linker sequence SGGGGSG
L3: linker sequence GAPLGGGGGGGGS
L4: linker sequence SGGSGGGGGGGGSG
L5: linker sequence SGGS
L6: linker sequence GGSGGSGGSG
C: cysteine residue.

FIG. 6: Selection of the best indel strategy: uptake of IDS.IGF2 indel variants into MPS II fibroblasts. Data represent mean±SEM and are analyzed with one-way ANOVA with Bonferroni's multiple testing correction. Significance is expressed relative to IDS. ***P<0.001, ****P<0.0001.

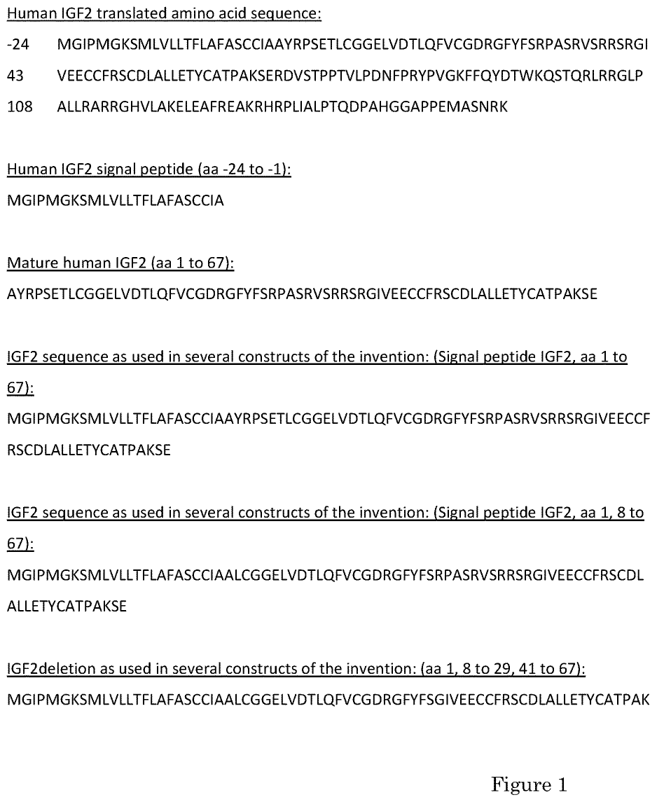

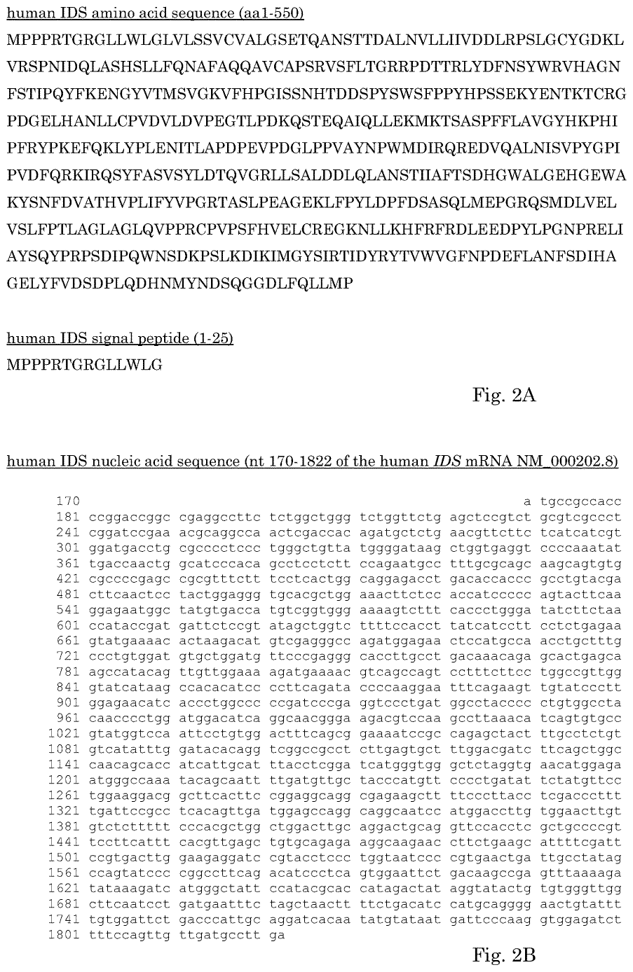

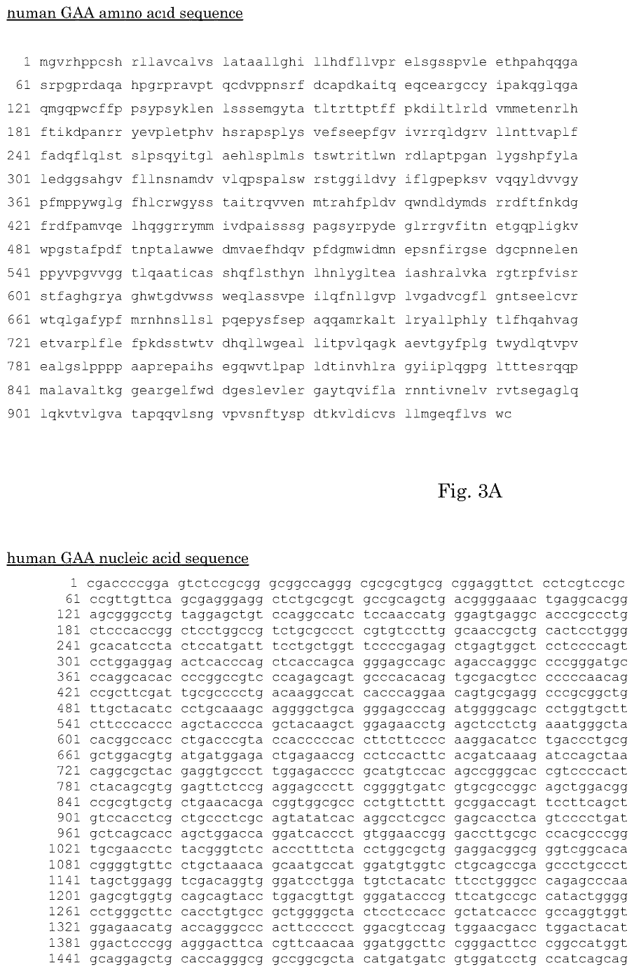

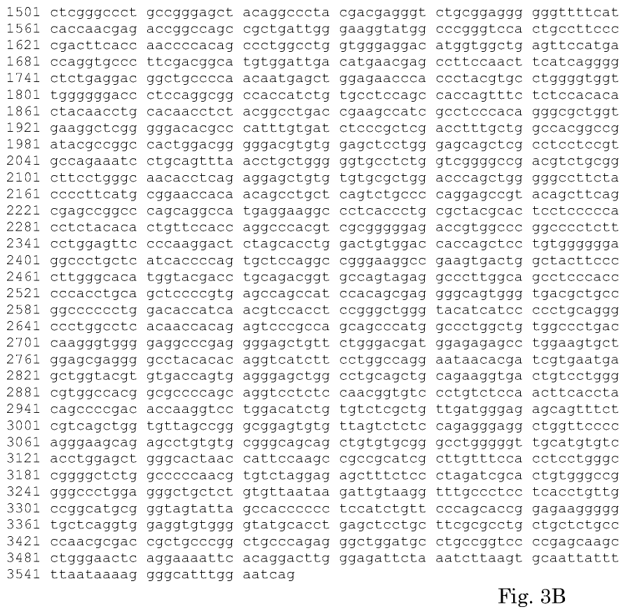

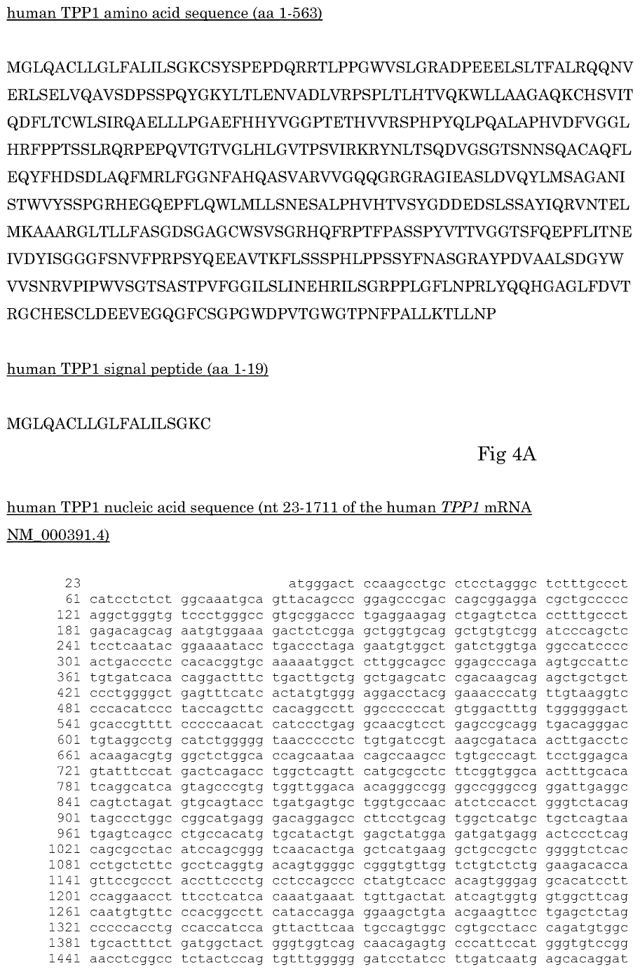

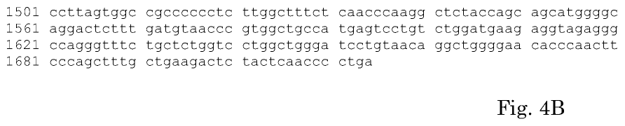

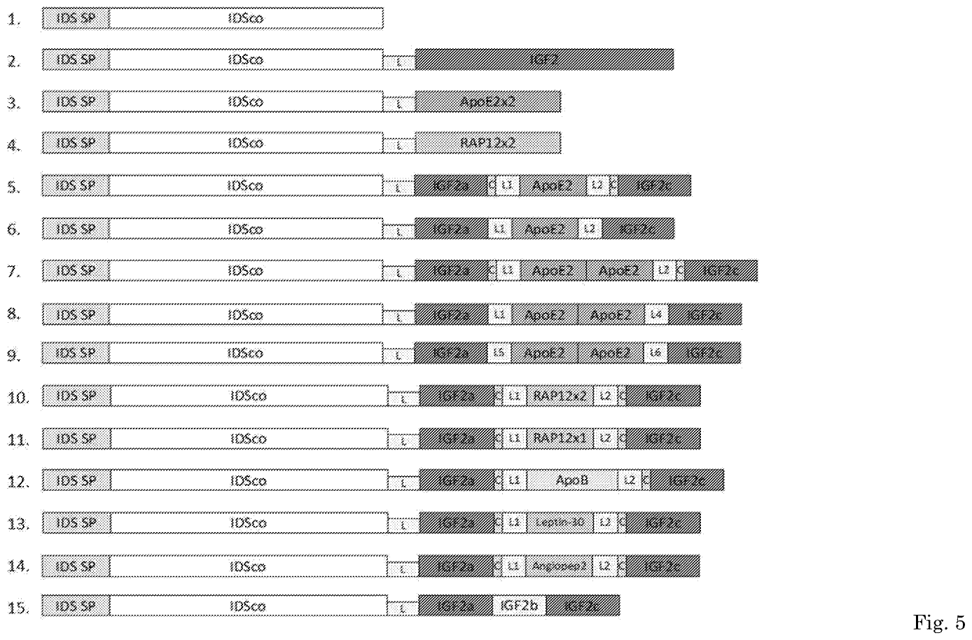

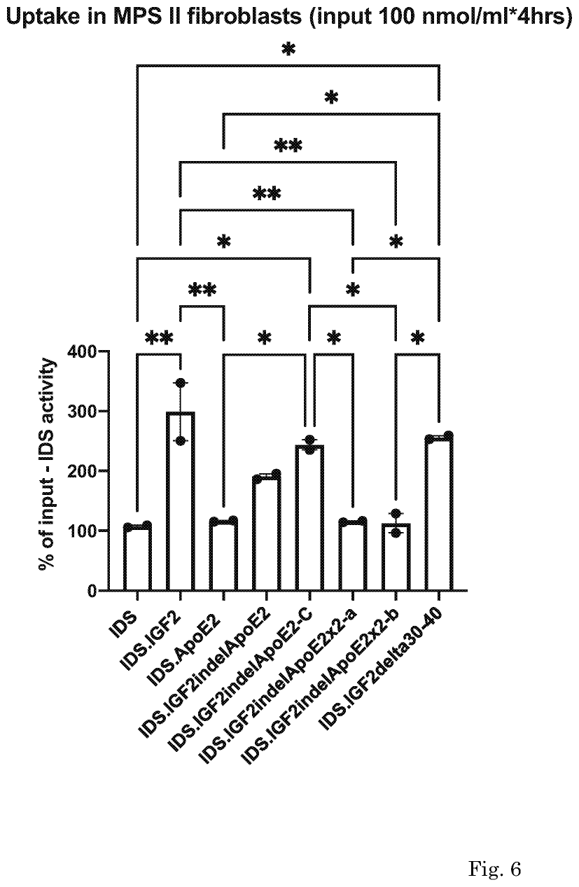

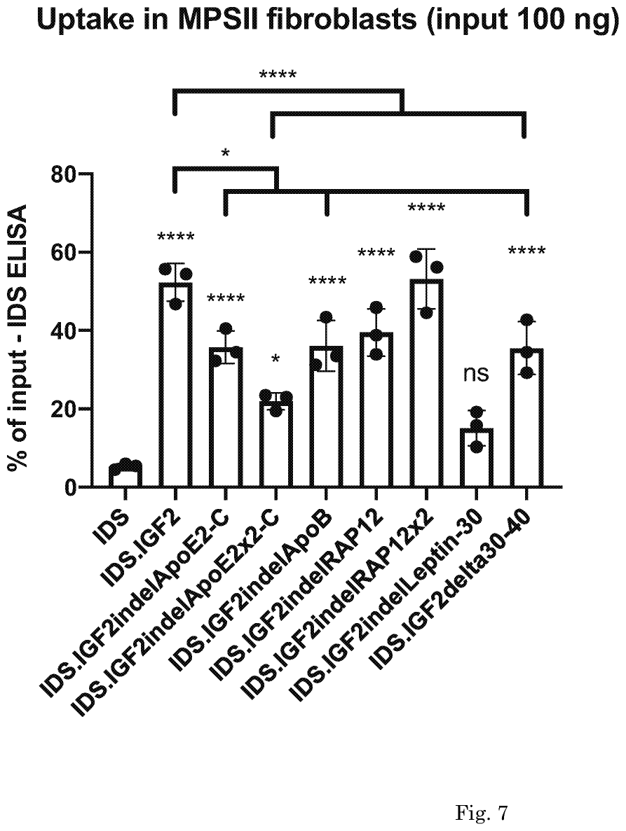

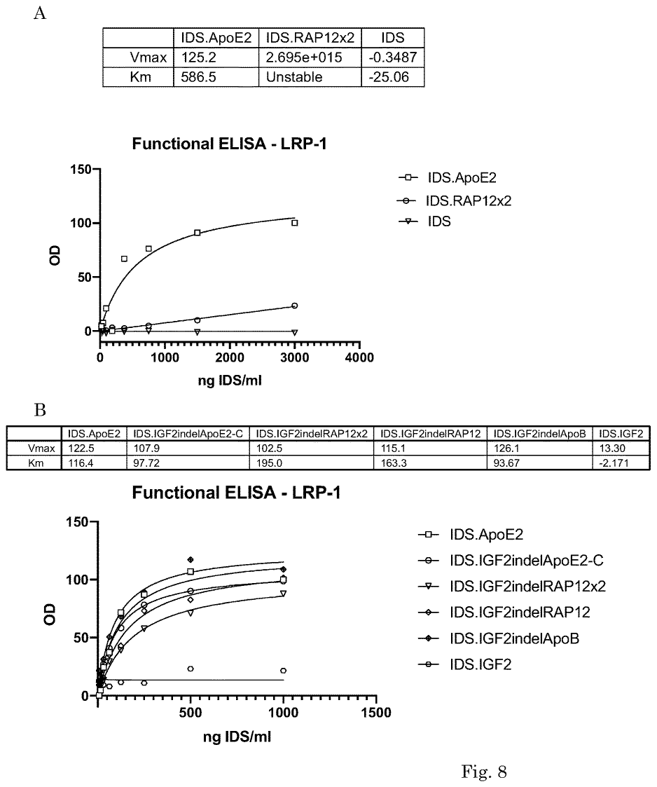

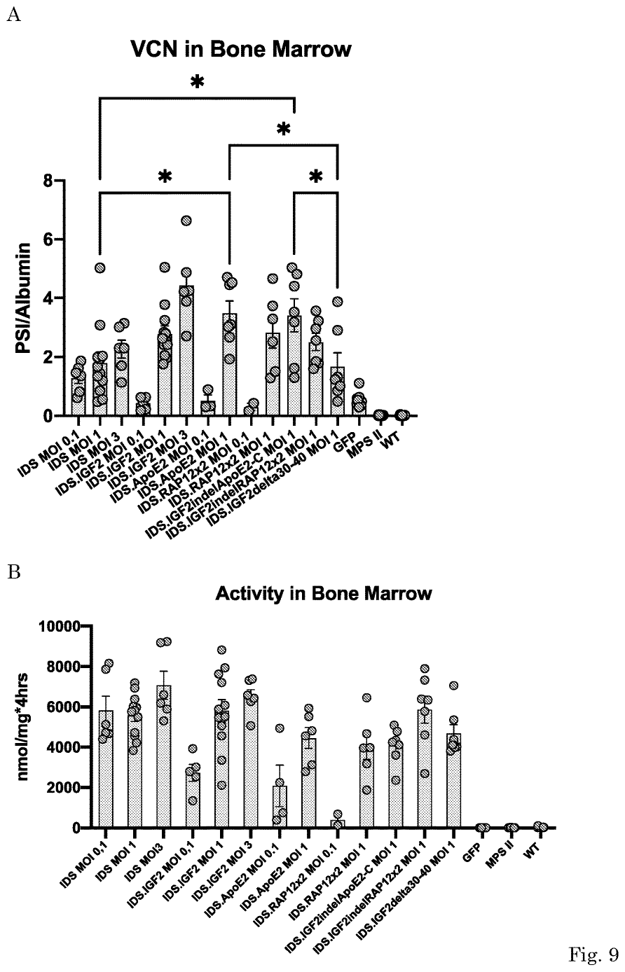

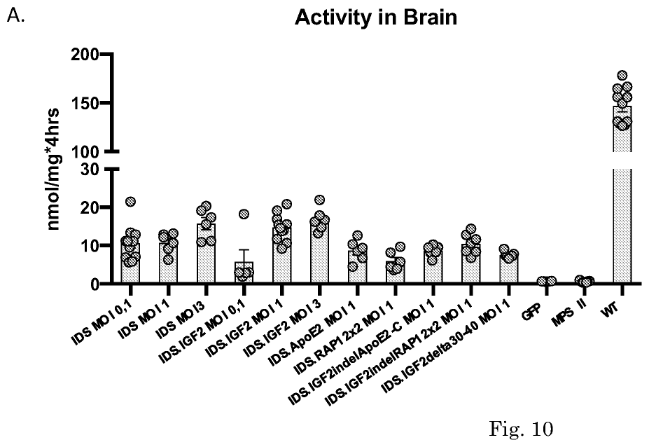

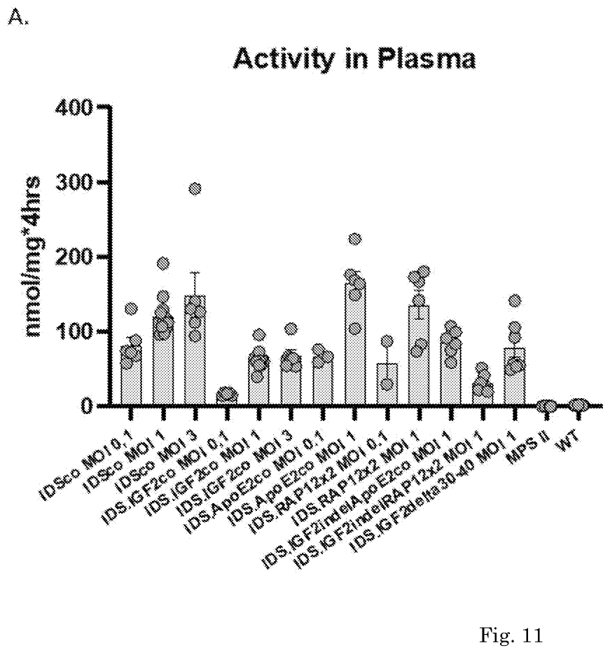

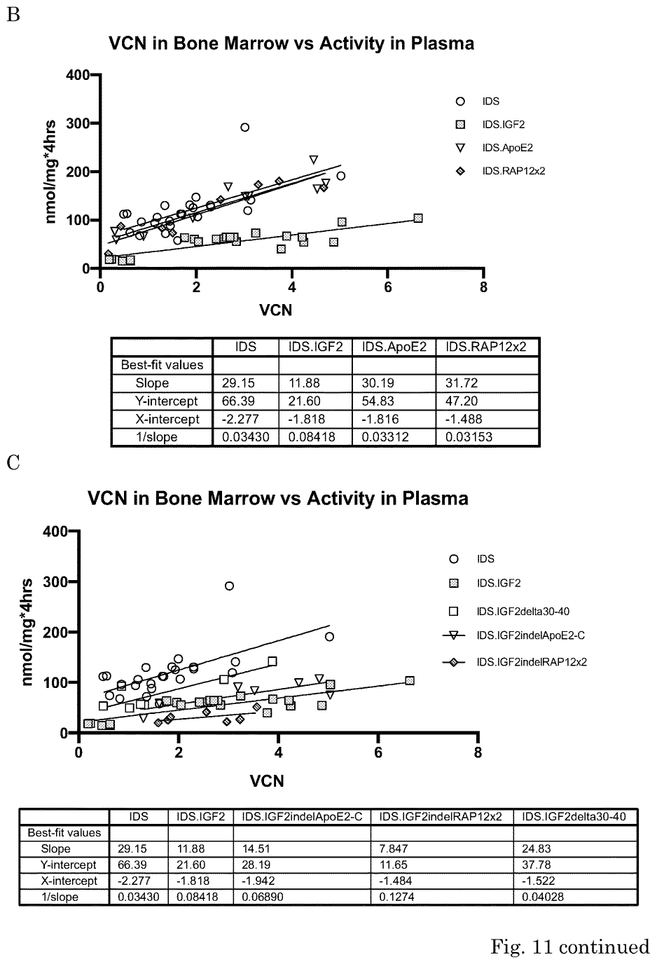

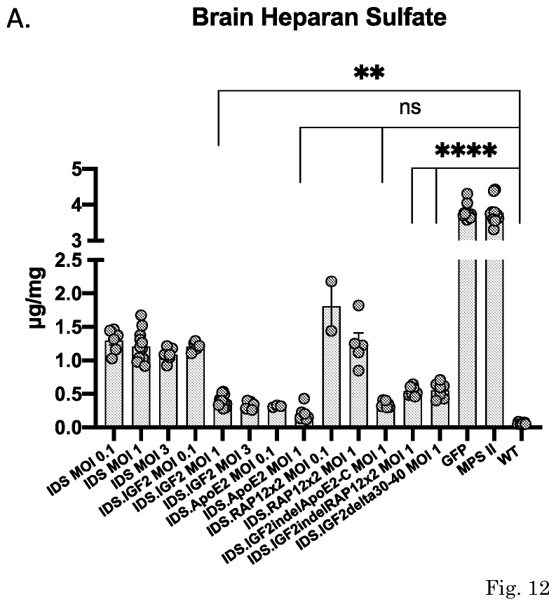

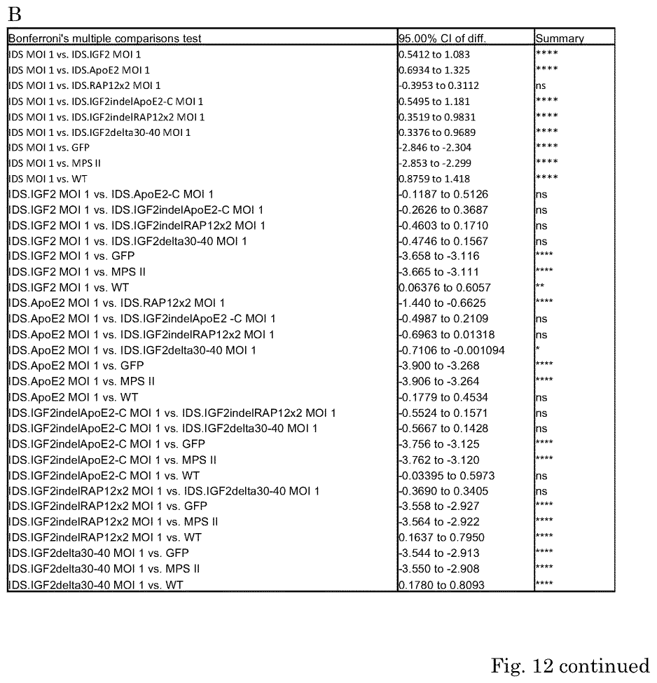

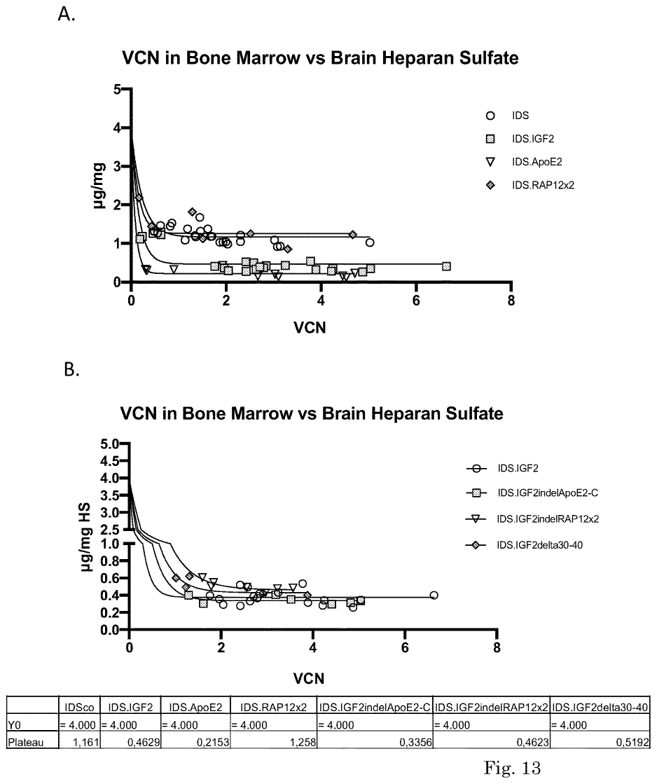

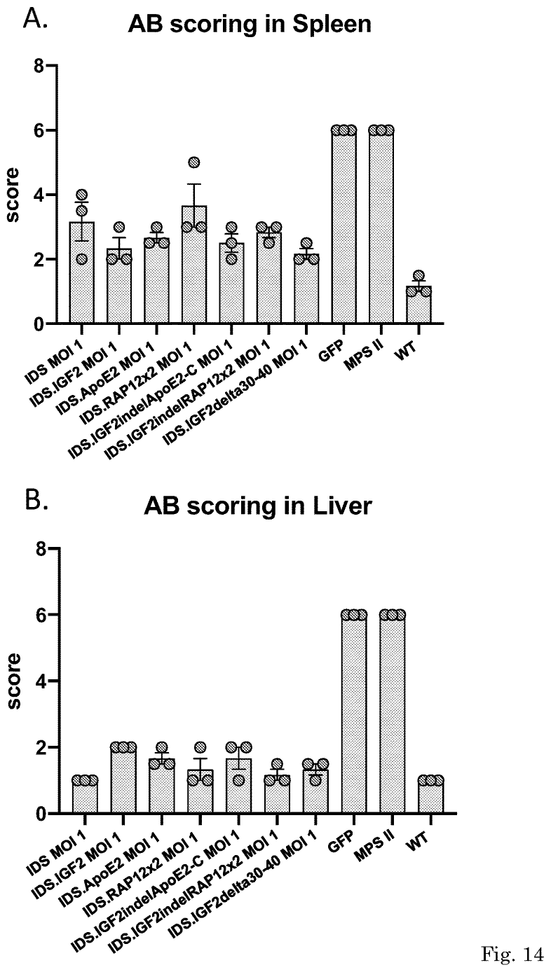

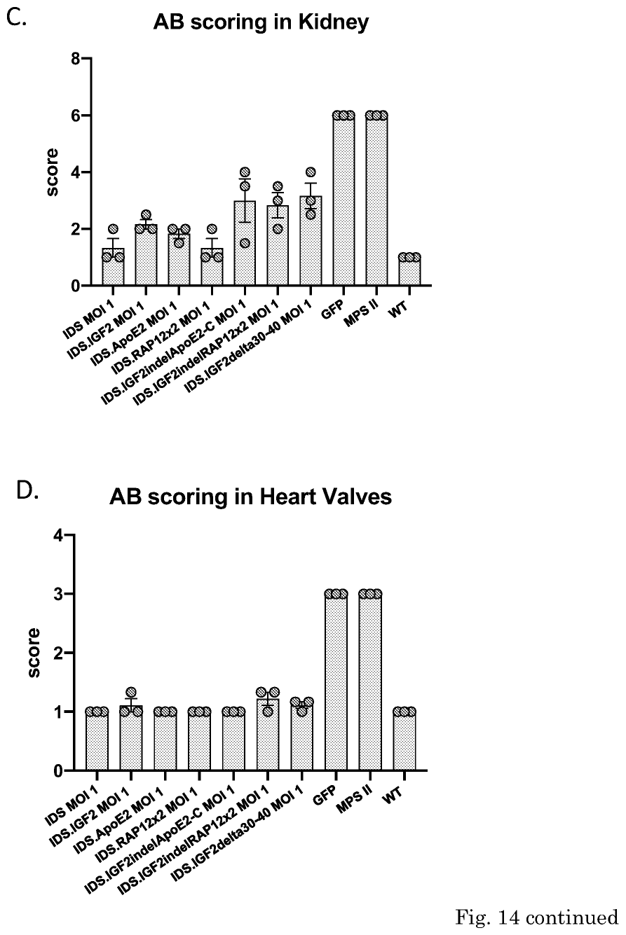

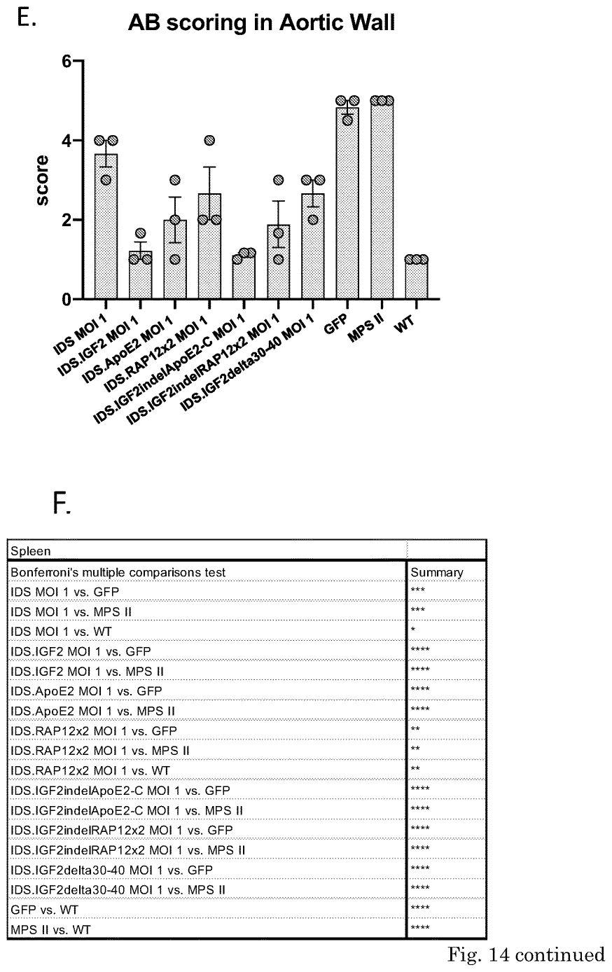

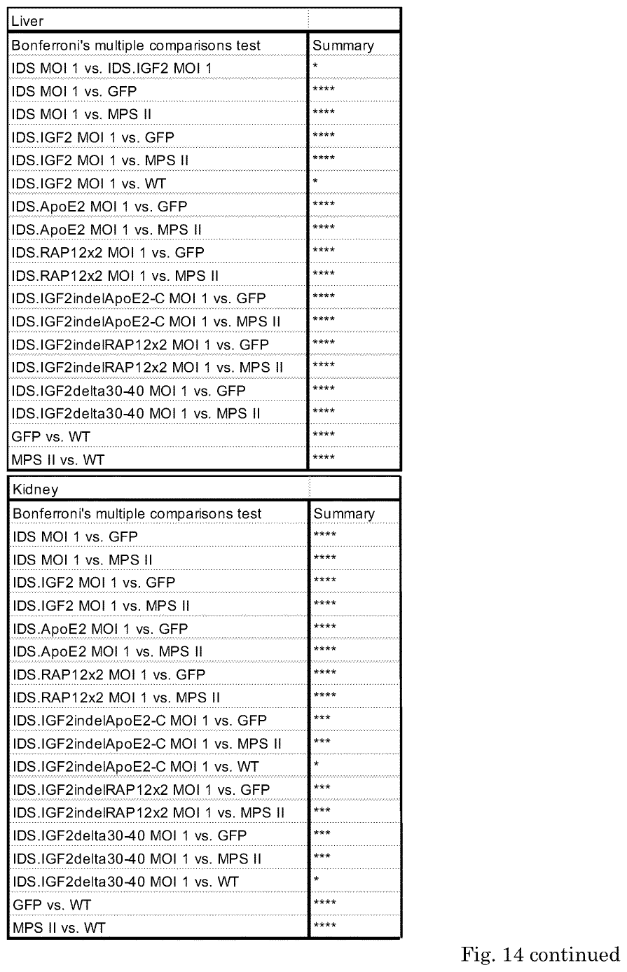

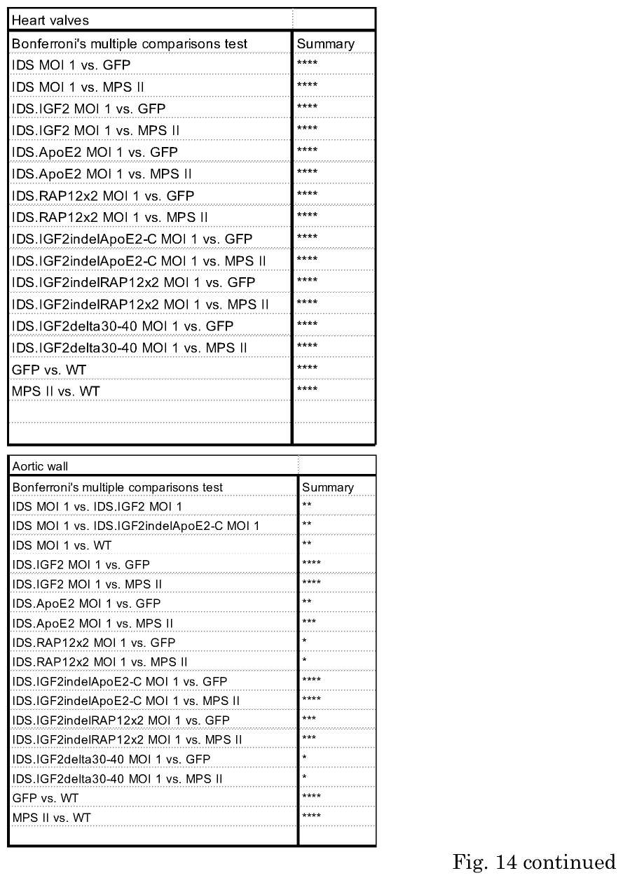

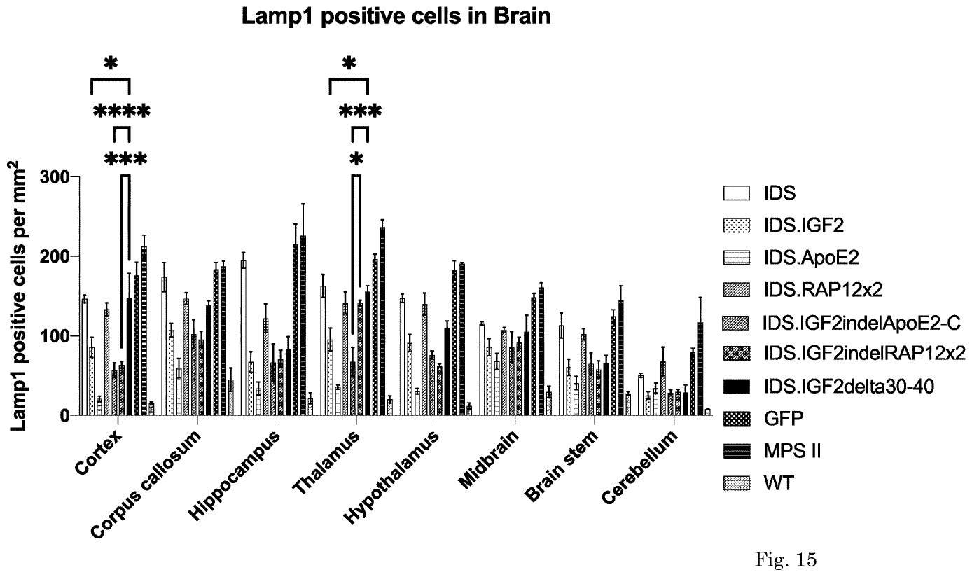

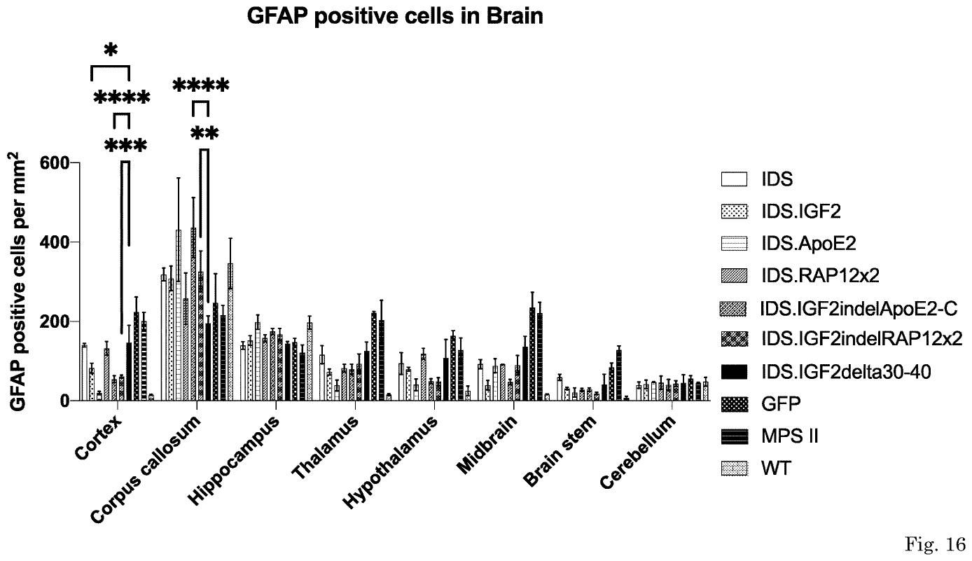

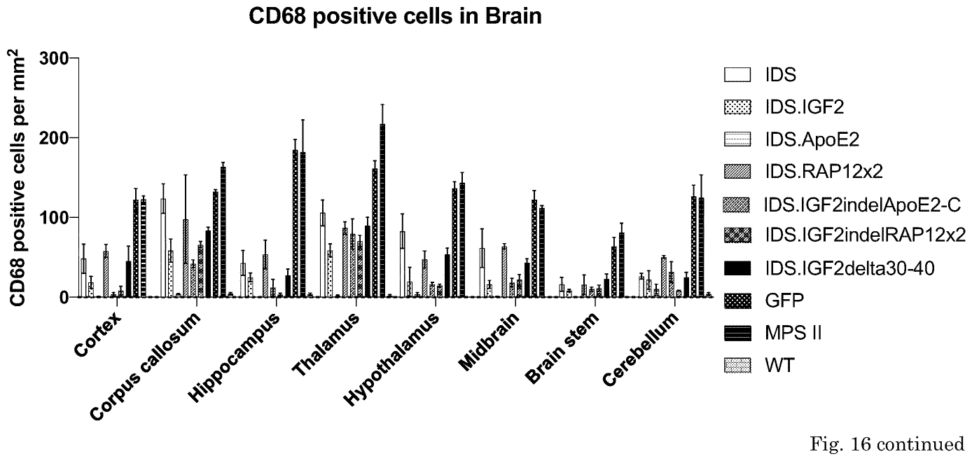

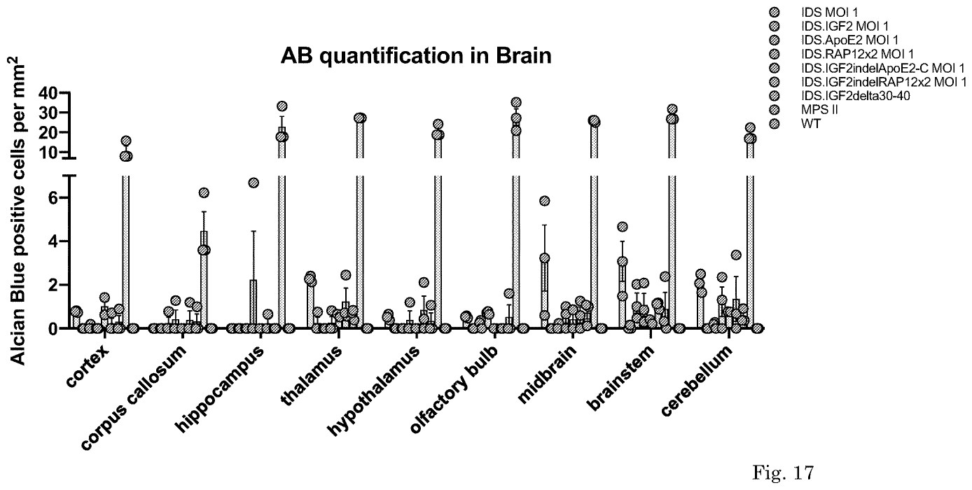

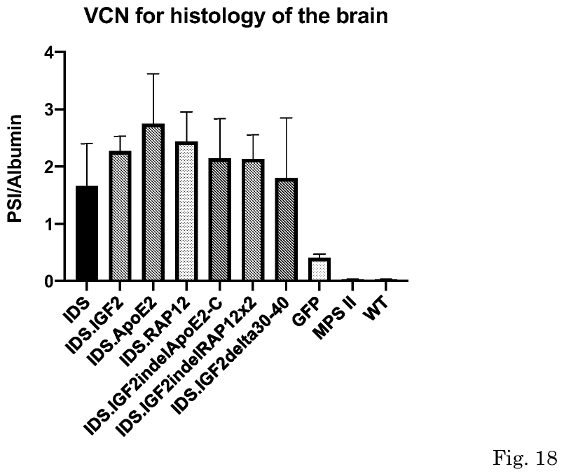

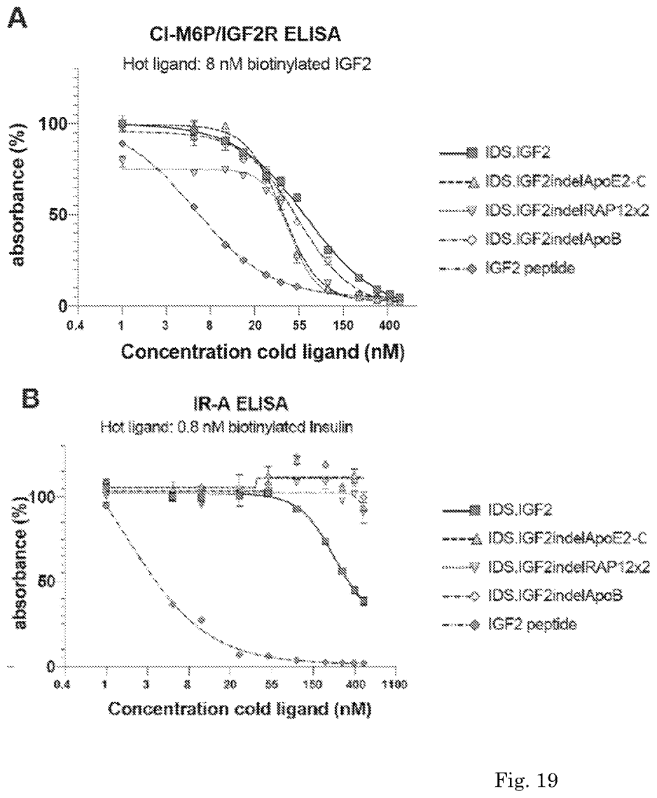

FIG. 7: Application of the optimal indel strategy containing cysteine residues flanking the insertion: uptake of IDS.IGF2 indel variants into MPS II fibroblasts. For sake of clarity, all the indel versions that were made after the IDS.IGF2indelAPoE2-C contain a double cysteine residue flanking the insertion, although this is not anymore indicated in the name. See description for FIG. 1.

Data represent mean±SEM and are analyzed with one-way ANOVA with Bonferroni's multiple testing correction. Significance is expressed relative to IDS. ns, not significant; ***P≤0.001, ****P≤0.0001.

FIG. 8: LRP-1 Functional ELISA. A. LRP-1 functional ELISA with IDS, IDS.ApoE2, IDS.RAP12x2. B. LRP-1 functional ELISA with IDS.ApoE2, IDS.IGF2indelRAP12x2, IDS.IGF2indelRAP12, IDS.IGF2indelApoE, IDS.IGF2indelApoB. Non-linear fit curves are shown. Km and Vmax values are shown.

FIG. 9: Hematopoietic stem cells (HSC)-mediated lentiviral gene therapy in MPS II mice results in supraphysiological IDS activity levels in bone marrow

2-5 months old CD45.1 ids/mice were sacrificed and Lineage cells were purified from bone marrow as previously described. Lineage cells were briefly cultured ex vivo and transduced with lentiviral vectors coding for construct 1-construct 9 or GFP as mock control at the indicated multiplicity of infection (MOI). 2-3 months old CD45.2 ids^{-/-} mice were preconditioned with total body irradiation at a dose of 9 Gy. 24 hr after preconditioning, mice were transplanted with 10\hat{ }6 cells. 6 months after gene therapy, mice were sacrificed and tissues were harvested for histological and molecular analysis. (A) average integrated vector copy number (VCN) in bone marrow. VCN was determined by qPCR as previously described. (B) IDS enzyme activity in bone marrow. Data in (A) and (B) represent mean±SEM and are analysed by one-way ANOVA with Bonferroni's multiple testing correction. *P≤0.05

FIG. 10: Gene therapy results in increased IDS activity levels in the brain. (A) IDS enzyme activity in brain 6 months after gene therapy. Data in (A) represent mean±SEM.

FIG. 11: Gene therapy results in supraphysiological IDS activity levels in plasma. (A,B) IDS enzyme activity in plasma 6 months after gene therapy. (B, C) VCN in brain is plotted against activity in plasma. Linear regression results are shown in (B, C). Data in (A) represent mean±SEM.

FIG. 12: Gene therapy results in reduction of brain heparan sulfate. (A) HPLC-MS quantification of brain heparan sulfate (HS) as previously described. Multiple comparison results are shown in (B). Data are analysed by one-way ANOVA with Bonferroni's multiple testing correction and are presented as mean±SEM. *P≤0.05, **P≤0.01, ****P≤0.0001.

FIG. 13: Relationship between VCN in bone marrow and HS levels in brain. (A, B) VCN in brain is plotted against brain HS. Non-linear regression results are shown.

FIG. 14: Gene therapy results in normalization of alcian blue staining in spleen, liver, kidney, heart valves and aortic wall. (A-F) Scoring of alcian blue staining in spleen, liver, kidney, heart valves and aortic wall, respectively. Scoring was performed by 2 independent operators blind to the experimental conditions. Scoring system is shown in table 1. n=3. AB=alcian blue. Data represent mean±SEM and are analysed by one-way ANOVA with Bonferroni's multiple testing correction. *P≤0.05, **P≤0.01, ***P≤0.001, ****P≤0.0001.

FIG. 15: Gene therapy results in a strong reduction of lysosomal pathology in the brain. Quantification of lamp1 deposition in brain 6 months after gene therapy. Lamp1 DAB staining was performed on brain sagittal sections and quantification was performed by counting the number of lamp1 positive cells in the areas shown. n=3; Data are mean±SEM. Data represent mean±SEM and are analysed by two-way ANOVA using brain region and treatment as categorical variable and with Bonferroni's multiple testing correction. *P≤0.05, ***P≤0.001, ****P≤0.0001.

FIG. 16 Gene therapy results in reduction of neuroinflammation in the brain. Quantification of lamp1 deposition in brain 6 months after gene therapy. GFAP (marker for astrocytes) and CD68 (marker for activated microglia) DAB staining was performed on brain sagittal sections and quantification was performed by counting the number of GFAP or CD68 positive cells in the areas shown. n=3; Data represent mean±SEM and are analysed by two-way ANOVA using brain region and treatment as categorical variable and with Bonferroni's multiple testing correction. *P≤0.05, **P≤0.01, ***P≤0.001, ****P≤0.0001.

FIG. 17: Gene therapy results in normalization of alcian blue staining in brain. Quantification of alcian blue staining in brain. Alcian blue staining was performed on brain sagittal sections and quantification was performed by counting the number of alcian blue positive cells in the areas shown. AB=alcian blue. Data are mean±SEM. n=3.

FIG. 18: Vector copy number of the mice used for histology of the brain. VCN was determined by qPCR as previously described.³ (B) IDS enzyme activity in bone marrow. Data are mean±SEM. n=3.

FIG. 19: Competitive IGF2R (A) and IR-A (B) ELISA with the indicated ligands at the indicated concentrations. Data represent means±SD. n=2 technical replicates per condition.

### EXAMPLES

### Materials and Methods

Uptake into MPS II Fibroblasts

### Input Based on Enzyme Activity

HEK 293T cells were transfected with the self-inactivating lentiviral pCCL transfer vector coding for constructs 1-15 as previously shown.³ 48 hours after transfection, medium supernatant containing the secreted IDS variants was collected and IDS concentration was determined using 4MU assay to determine IDS activity. 100 nmol/ml*4 hrs of IDS were incubated with MPS II fibroblasts for 18 hours. After washing with PBS, captured IDS was measured in cell lysate using IDS sandwich ELISA. Results are shown in FIG. 6.

In other experiments, HEK 293T cells were transfected with the self-inactivating lentiviral pCCL transfer vector coding for constructs 1-15 as previously shown.³ 48 hours after transfection, medium supernatant containing the secreted IDS variants was collected and IDS concentration was determined using sandwich IDS ELISA.⁴ 100 ng of IDS were incubated with MPS II fibroblasts for 18 hours. After washing with PBS, captured IDS was measured in cell lysate using IDS sandwich ELISA. Results are shown in FIG. 7.

### LRP-1 Functional ELISA

HEK 293T cells were transfected as previously shown.³ 48 hours after transfection, medium supernatant containing the secreted IDS variants was collected and IDS concentration was determined using sandwich IDS ELISA (DY2449, R&D). Human recombinant LRP-1 receptor (5395-L4; R&D) was immobilized with human anti-IgG antibody (62-8400; Invitrogen) on Nunc-immuno Microwell plates M9410. Medium supernatant containing (A) 3, 1.5, 0.75, 0.375, 0.1875, 0.09375, 0.046875, 0.0234375 μg/ml of IDS or (B) 1, 0.5, 0.25, 0.125, 0.0625, 0.03125, 0.015635 or 0.0078125 μg/ml of IDS was added to the LRP-1 coated wells. LRP-1-bound IDS protein was detected using biotinylated anti-IDS capture antibody and HRP-streptividin (DY2449, R&D). Optical density was determined using a microplate reader (Varioskan, Thermo Electron Corporation) set at 450 nm and corrected for optical imperfections measured at 570 nm.

### Lentiviral Vector Construction and Production

Codon-optimized human IDS was cloned into the third generation self-inactivating (SIN) lentiviral vector pCLL.PPT.MND.RAG1.WPRE.SIN (pCCL.MND.coRAG1, kindly provided by Dr. Frank Staal; Garcia-Perez et al. Mol Ther Methods Clin Dev. 2020 Mar. 31; 17:666-682. doi: 10.1016/j.omtm.2020.03.016) by replacing the RAG1 gene using BamHI and SalI restriction sites to generate pCLL.PPT.MND.IDSco.WPRE.SIN (IDSco). Codon-optimized cassettes coding for tags described in FIG. 5, constructs 2-15 were cloned into the IDSco backbone after double digestion using SbfI and SalI. Lentiviral vectors were generated in HEK 293T cells by calcium phosphate transfection with the 3^{rd} generation lentiviral vector packaging plasmids pMDL-g/pRRE, pMD2-VSVg, and pRSV-Rev (Dull et al. 1998 J. Virol. 72, 8463; Zufferey et al. 1998 J. Virol. 72, 9873-9880). Virus concentration was performed by ultracentrifugation (Beckman, SW32Ti rotor) at 20,000 rpm for 2 hours at 4° C. and titration was performed in HeLa cells by quantitative polymerase chain reaction (qPCR) with primers targeting the U3 and Psi sequences of HIV. A standard curve was prepared using transduced HeLa with on average 1 copy of integrated lentiviral vector per genome. Viral titre concentration was determined as the average VCNs multiplied by the cell number and fold dilution.

### Hematopoietic Stem Cells (HSC)-Mediated Lentiviral Gene Therapy in MPS II Mice

2-5 months old CD45.1 ids^{-/-} mice were sacrificed and Lineage cells were purified from bone marrow as previously described.^{2,5} Lineage cells were briefly cultured ex vivo and transduced with lentiviral vectors coding for construct 1-construct 9 or GFP as mock control at the indicated multiplicity of Infection (MOI). 2-3 months old CD45.2 ids/mice were preconditioned with total body irradiation at a dose of 9 Gy. 24 hr after preconditioning, mice were transplanted with 10\hat{ }6 cells. 6 months after gene therapy, mice were sacrificed and tissues were harvested for histological and molecular analysis.

### Immunohistochemistry

Spleen, liver, kidney, heart and brain were fixed in metacharn solution (30% chloroform, 60% methanol, 10% acetic acid) for 24 hours. Samples were embedded into paraffin in a leica tissue processor TP1020 Histokinette and sectioned at a thickness of 8 or 10 μm. Alcian blue staining was performed as shown by Alliegro and colleagues⁶ and scored as shown in table 1. Dab staining was performed as shown by Doyle and colleagues.⁷

TABLE 1
Scoring of alcian blue staining.
Semi-quantitative scoring of alcian blue staining
in spleen, liver and kidney

| Score | Rules |
|---|---|
| 1 | 0 Alcian blue positive cells in 5 random fields |
| 2 | A total of 1-5 alcian blue positive cells in 5 random fields |
| 3 | A total of 5-10 alcian blue positive cells in 5 random fields |
| 4 | A total of 10-15 alcian blue positive cells in 5 random fields |
| 5 | A total of 15-30 alcian blue positive cells in 5 random fields |
| 6 | more than 100 Alcian blue positive cells in 1 random field |

Scoring of alcian blue staining in heart valves

| Score | Rules: |
|---|---|
| 1 | Weak alcian blue staining |
| 2 | Mild alcian blue staining |
| 3 | Strong alcian blue staining |

Scoring of alcian blue staining in aortic wall

| Score | Rules |
|---|---|
| 1 | No alcian blue staining |
| 2 | Alcian blue staining in less than 5% of the aortic wall area |
| 3 | Alcan blue staining in less than 30% of the aortic wall area |
| 4 | Alcian blue staining in less than 60% of the aortic wall area |
| 5 | Strong alcian blue staining in all the aortic wall area |

### Molecular Analysis

VCN in bone marrow was determined as previously described.² HPLC-MS quantification of brain heparan sulfate was performed as previously described.¹ Quantification of IDS enzyme activity was performed as previously described.⁸

### Results

### Construction of 3^{rd} Generation Lentiviral Vectors Coding for IDS and Tagged Variants of IDS

The constructs are shown in FIG. 5. All the constructs are cloned into a pCCL 3rd generation lentiviral vector.
Construct 1/IDS codes for untagged IDS.
Construct 2/IDS.IGF2 codes for IDS tagged C-terminally with aa 1, 8-67 of the mature human IGF2 (aa 1 is the first aa after the signal peptide).
Construct 3./IDS.ApoE2 codes for IDS tagged C-terminally with aa (141-149)x2 of the human apolipoprotein E (aa 1 is the first aa after the signal peptide).
Construct 4./IDS.RAP12x2 codes for IDS tagged C-terminally with aa (251-262) of the human receptor associated protein (RAP) in a tandem repeat separated by a glycine residue (aa 1 is the first aa after the signal peptide).
Construct 5./IDS.IGF2indelApoE-C codes for IDS tagged C-terminally with aa 1, 8-28, 42-67 of the mature human IGF2 (aa 1 is the first aa after the signal peptide). The sequence LRKLRKRLL, corresponding to aa 141-149 of the human apolipoprotein E, and flanked by the sequences CSGGGG (N-terminal) and SGGGGSGC (C-terminal) has been inserted between aa 28 and 42 of the human IGF2.
Construct 6./IDS.IGF2indelApoE2 codes for IDS tagged C-terminally with aa 1, 8-28, 42-67 of the mature human IGF2 (aa 1 is the first aa after the signal peptide). The sequence LRKLRKRLL, corresponding to aa 141-149 of the human apolipoprotein E, and flanked by the sequences SGGGG (N-terminal) and SGGGGSG (C-terminal) has been inserted between aa 28 and 42 of the human IGF2.
Construct 7./IDS.IGF2indelApoE2x2-C codes for IDS tagged C-terminally with aa 1, 8-28, 42-67 of the mature human IGF2 (aa 1 is the first aa after the signal peptide). The sequence LRKLRKRLLLRKLRKRLL, corresponding to aa 141-149 of the human apolipoprotein E, and flanked by the sequences CSGGGG (N-terminal) and SGGGGSGC (C-terminal) has been inserted between aa 28 and 42 of the human IGF2.
Construct 8./IDS.IGF2indelApoE2x2-a codes for IDS tagged C-terminally with aa 1, 8-28, 42-67 of the mature human IGF2 (aa 1 is the first aa after the signal peptide). The sequence LRKLRKRLLLRKLRKRLL, corresponding to aa 141-149 of the human apolipoprotein E, and flanked by the sequences SGGGG (N-terminal) and SGGSGGGGSGGGGSG (C-terminal) has been inserted between aa 28 and 42 of the human IGF2.
Construct 9./IDS.IGF2indelApoE2x2-b codes for IDS tagged C-terminally with aa 1, 8-28, 42-67 of the mature human IGF2 (aa 1 is the first aa after the signal peptide). The sequence LRKLRKRLLLRKLRKRLL, corresponding to aa 141-149 of the human apolipoprotein E, and flanked by the sequences SGGS (N-terminal) and GGSGGSGGSG (C-terminal) has been inserted between aa 28 and 42 of the human IGF2.
Construct 10./IDS.IGF2indelRAP12x2 codes for IDS tagged C-terminally with aa 1, 8-28, 42-67 of the mature human IGF2 (aa 1 is the first aa after the signal peptide). The sequence EAKIEKHNHYQKGEAKIEKHNHYQK, where sequence EAKIEKHNHYQK is aa 251-262 of the human Receptor Associated Protein (RAP), and flanked by the sequences CSGGGG (N-terminal) and SGGGGSGC (C-terminal) has been inserted between aa 28 and 42 of the human IGF2.
Construct 11./IDS.IGF2indelRAP12 codes for IDS tagged C-terminally with aa 1, 8-28, 42-67 of the mature human IGF2 (aa 1 is the first aa after the signal peptide). The sequence EAKIEKHNHYQK, consisting of aa 251-262 of the human Receptor Associated Protein (RAP), and flanked by the sequences CSGGGG (N-terminal) and SGGGGSGC (C-terminal) has been inserted between aa 28 and 42 of the human IGF2.
Construct 12./IDS.IGF2indelApoB codes for IDS tagged C-terminally with aa 1, 8-28, 42-67 of the mature human IGF2 (aa 1 is the first aa after the signal peptide). The sequence SSVIDALQYKLEGTTRLTRKRGLKLATALSLSNKFVEGS, corresponding to aa 3371-3409 of the human apolipoprotein B, and flanked by the sequences CSGGGG (N-terminal) and SGGGGSGC (C-terminal) has been inserted between aa 28 and 42 of the human IGF2.
Construct 13./IDS.IGF2indelLeptin-30 codes for IDS tagged C-terminally with aa 1, 8-28, 42-67 of the mature human IGF2 (aa 1 is the first aa after the signal peptide). The sequence YQQILTSMPSRNVIQISNDLENLRDLLHVL is flanked by the sequences CSGGGG (N-terminal) and SGGGGSGC (C-terminal) has been inserted between aa 28 and 42 of the human IGF2.
Construct 14./IDS.IGF2indelAngiopep2 codes for IDS tagged C-terminally with aa 1, 8-28, 42-67 of the mature human IGF2 (aa 1 is the first aa after the signal peptide). The sequence TFFYGGSRGKRNNFKTEEY is flanked by the sequences CSGGGG (N-terminal) and SGGGGSGC (C-terminal) has been inserted between aa 28 and 42 of the human IGF2.
Construct 15./IDS.IGF2delta30-40 codes for IDS tagged C-terminally with aa 1, 8-29, 41-67 of the mature human IGF2 (aa 1 is the first aa after the signal peptide).
   Uptake into MPS II Fibroblasts

The IGF2indel design was optimized by tagging IDS C-terminally with one of four IGF2indel variants carrying an insertion of ApoE2, a peptide able to bind members of the LDL receptor family (LDLrf) (FIG. 6). The four IGF2indel variants differed for the length and composition of the linkers flanking the ApoE2 insertion (different combinations used in the 4 constructs; see Brief description of the drawings, page 39), and the absence or presence of a Cysteine residue at the sides of the insertion.
a. IDS.IGF2indelApoE2-C(construct 5 in FIG. 5): one ApoE2 repeat; Cysteine at both sides of the insertion; linkers L1 and L2;
b. IDS.IGF2indelApoE2 (construct 6 in FIG. 5): one ApoE2 repeat; no Cysteine; linkers L1 and L2;
c. IDS.IGF2indelApoE2x2-a (construct 8 in FIG. 5): two tandem ApoE2 repeats; no Cysteine; linkers L1 and L4;
d. IDS.IGF2indelApoE2x2-b (construct 9 in FIG. 5): two tandem ApoE2 repeats; no Cysteine; linkers L5 and L6.

The IGF2indel designs were evaluated by comparing their uptake values into patient-derived MPS II fibroblasts with those of IGF2-tagged IDS (IDS.IGF2), IDS tagged with an IGF2 tag carrying a deletion of AA 30-40 (IDS.IGF2delta30-40) and ApoE2-tagged IDS (in a tandem repeat, IDS.ApoE2).

As shown in FIG. 6, IDS.IGF2 showed slightly higher uptake values compared to IDS.IGF2delta30-40, and ˜3-fold higher uptake levels compared to IDS.ApoE2. IGF2indels with a tandem repeat of ApoE2 (IDS.IGF2indelApoE2x2-a and IDS.IGF2indelApoE2x2-b) resulted in uptake levels comparable to IDS.ApoE2 and ˜3 fold lower compared to IDS.IGF2. In contrast, the insertion of a single repeat of ApoE2 resulted in uptake levels which were slightly lower (IDS.IGF2indelApoE2) or comparable (IDS.IGF2indelApoE2-C) to IDS.IGF2delta30-40.

In particular, the inclusion of a cysteine residue at both sides of the insertion appeared to be beneficial during uptake into MPS II fibroblasts (FIG. 6). For this reason, we selected the design of IDS.IGF2indelApoE2-C-characterized by the “SGGG” linker at the N-terminus of the insertion, the linker “SGGGGSG” at the C-terminus of the insertion, and a cysteine residue at the N-terminus and C-terminus of the two linkers—to further test IGF2indel variants with additional inserted sequences (FIG. 7).

To evaluate the applicability of the chosen IGF2indel design for a modular switch of epitopes, we substituted the ApoE2 sequence in IDS.IGF2indelApoE2-C with alternative sequences (FIG. 7). Furthermore, to investigate the cargo capacity of the chosen IGF2indel design, we specifically chose epitopes of varying lengths, ranging from as short as the 9 AA of the ApoE2, to as long as the 39 AA of the ApoB. This resulted in 6 IGF2indel versions containing 6 different insertions:
ApoE2 (IDS.IGF2indelApoE2-C),
ApoE2x2 (a tandem repeat of the ApoE2, IDS.IGF2indelApoE2x2-C),
RAP12 (IDS.IGF2indelRAP12),
RAP12x2 (a tandem repeat of the RAP12 spaced by a glycine residue, IDS.IGF2indelRAP12x2),
ApoB (IDS.IGF2indelApoB), and
Leptin-30 (IDS.IGF2indelLeptin-30).

All these constructs contain a cysteine residue at the N-terminus and C-terminus of the two linkers flanking the insert.

Next, we tested these versions during uptake into MPS II fibroblasts. As shown in FIG. 7, all the constructs tested provide improved uptake compared to untagged-IDS. IDS.IGF2indelRAP12x2 showed uptake values comparable to IDS.IGF2, while IDS.IGF2indelApoE2-C, IDS.IGF2indelRAP12, and IDS.IGF2indelApoB resulted in values slightly decreased compared to IDS.IGF2, and comparable to IDS.IGF2delta30-40. We selected IDS.IGF2indelApoE2, IDS.IGF2indelRAP12x2 and IDS.IGF2indelApoB for further in vitro tests (FIG. 19).

To investigate the binding to the CI-M6P/IGF2R, we conducted a competitive binding assay using domain 11 of the CI-M6P/IGF2R, known to specifically bind IGF2. In this assay, we used a fixed concentration of biotinylated IGF2 (at 8 nM; referred to as hot ligand) and competed with a range of concentrations (from 1 to 500 nM; referred to as cold ligand) of:
IDS.IGF2,
IDS.IGF2indelApoE2-C,
IDS.IGF2indelRAP12x2,
IDS.IGF2indelApoB or
IGF2 peptide carrying a mutation of AA 1-6 (A1-6.IGF2 peptide).

Binding of IGF2 to the CI-M6P/IGF2R differed when used as a peptide or when tagged to IDS, with IDS.IGF2 showing a ˜12.3-times increased IC₅₀ values compared to the untagged Δ1-6.IGF2 peptide (IC₅₀ IDS.IGF2: 61.69 nM; IC₅₀ Δ1-6.IGF2 peptide: 5.008 nM) (FIG. 19A), suggesting that binding of IGF2 to the CI-M6P/IGF2R is partially inhibited when tagged to IDS.

The examined IDS.IGF2indel versions showed a slightly higher affinity for the CI-M6P/IGF2R compared to IDS.IGF2, with IC₅₀ of IDS.IGF2indelApoE2, IDS.IGF2indelRAP12x2 and IDS.IGF2indelApoB being ˜1.69, 1.44 and 1.21-times lower than the IC₅₀ of IDS.IGF2, respectively (IC₅₀ IDS.IGF2indelApoE2: 36.45 nM; IC₅₀ IDS.IGF2indelRAP12x2: 42.92 nM; IC₅₀ IDS.IGF2indelApoB: 50.92 nM) (FIG. 19 A).

Binding to the insulin receptor (IR) was also examined. Similarly to the competitive CI-M6P/IGF2R ELISA assay, we employed a fixed concentration of biotinylated insulin (at 0.8 nM; referred to as hot ligand) and competed it against a series of concentrations of IDS.IGF2indel versions, as well as IDS.IGF2 and Δ1-6.IGF2 peptide (from 1 to 500 nM; referred to as cold ligand) for binding to the IR isoform-A (IR-A)).

Similar to the observations made during the CI-M6P/IGF2R ELISA assay, we noticed an impact of IDS tagging on binding of IGF2 to the IR-A (FIG. 19B). Specifically, IDS.IGF2 bound the IR-A with an affinity that was ˜150-fold lower than the affinity of Δ1-6.IGF2 peptide (IC₅₀ IDS.IGF2: 250.7 nM; IC₅₀ Δ1-6.IGF2 peptide: 1.607 nM), suggesting a partial hindrance of IGF2 binding to IR-A when tagged to IDS.

In contrast, none of the tested IDS.IGF2indel versions exhibited an appreciable binding to the IR-A, as evidenced by the complete lack of competition against biotinylated insulin.

### LRP-1 Functional ELISA

Binding of IDS.RAP12x2 to the LRP-1 receptor is less efficient than binding of IDS.IGF2indelRAP12x1 and IDS.IGF2indelRAP12x2 to the same receptor, which might be caused by inaccessibility of the RAP12 tag for binding to LRP-1 when present in IDS.RAP12 (FIG. 8A). IDS.IGF2 indel versions containing either ApoE2, RAP12 or ApoB tags are able to bind the LRP-1 receptor with an affinity comparable to IDS.ApoE2. IDS.IGF2 does not bind efficiently to the LRP-1 receptor (FIG. 8B).

The insertion of RAP12 as indel version within the accessible loop structure of the IGF2 tag likely provides accessibility of the RAP12 tag to enable binding to the LRP-1 receptor. IDS.IGF2indelApoE2-C and IDS.IGF2indelApoB have superior affinity for the LRP-1 receptor compared to IDS.IGF2indelRAP12 and IDS.IGF2indelRAP12x2

### Hematopoietic Stem Cells (HSC)-Mediated Lentiviral Gene Therapy in MPS II Mice

As demonstrated in FIG. 9, HSC-mediated lentiviral gene therapy in MPS II mice results in supraphysiological IDS activity levels in bone marrow. IDS activity in brain is comparable between the different lentiviral vectors tested (FIG. 10). IDS activity in plasma is lower after gene therapy with IDS.IGF2 or IDS.IGF2indel versions compared to IDS, IDS.ApoE2, IDS.RAP12x2, and IDS.IGF2delta30-40 (FIG. 11). This lower IDS activity in plasma is likely due to the higher uptake of the IDS.IGF2 and IDS.IGF2indel versions compared to the IDS and IDS.ApoE2 constructs, as shown in FIG. 6 and FIG. 7, which might reduce the plasma half-life of these constructs.

In brain, IDS.IGF2 and IDS.ApoE2 are comparable in reducing heparan sulfate (HS), the compound that accumulates in Hunter disease. IDS.IGF2, IDS.ApoE2, IDS.IGF2indelApoE2-C, IDS.IGF2indelRAP12x2 and IDS.IGF2delta30-40 are the most effective LV treatments for reducing HS in the brain (see FIG. 12). IDS.IGF2indelApoE2-C plateaus at a lower brain HS level compared to both IDS.IGF2 and IDS.IGF2delta30-40 (FIG. 13, lower panel; plateaus levels 0.34, 0.46 and 0.52 respectively). IDS.IGF2indelRAP12x2 shows lower efficacy in treating brain HS compared to IDS.IGF2indelApoE2-C, in line with the lower affinity of IDS.IGF2indelRAP12x2 for the LRP-1 of the IDS.IGF2indelRAP12x2 construct compared to IDS.IGF2indelApoE2-C(and shown in FIG. 8).

Alcian blue binds specifically to acid mucines like heparan and dermatan sulfate glycosaminoglycans. Alcian blue staining was performed to visualize accumulation of glycosaminoglycans in in spleen, liver, kidney, heart valves and aortic wall. All indel vectors and other vectors are able to normalize alcian blue positive staining in spleen, liver, kidney and heart valves (see FIG. 14). As demonstrated in FIG. 17, IDS.IGF2 indel vectors, as well as IDS.IGF2 and IDS.ApoE2 constructs were more effective than untagged IDS in normalizing AB staining in brain.

Lysosomal-associated membrane protein 1 (Lamp1) is a marker of lysosomes. In MPS II mouse brain there is a widespread increased in Lamp1 deposition (FIG. 15). Lentiviral gene therapy resulted in small (IDS and IDS.RAP12x2 treatment) to strong (IDS.IGF2, IDS.IGF2indel versions, IDS.IGF2delta30-40 and IDS.ApoE2) reduction of Lamp1 deposition in the brain of MPS II mice. IDS.IGF2 indel versions and IDS.IGF2 showed a comparable efficacy. In cortex, IGF2 indel versions showed a better correction of Lamp1 pathology compared to IDS.IGF2delta30-40, while in thalamus IDS.IGF2 and IDS.IGF2indelApoE2-C showed a better performance compared to IDS.IGF2delta30-40.

GFAP is marker for activated astrocytes, a hallmark of neuroinflammation. GFAP deposition is increased in cortex, thalamus, hypothalamus, midbrain, brainstem and cerebellum of MPS II mice compared to WT, while it is reduced in corpus callosum and hippocampus (FIG. 16). IDS.IGF2, IDS.IGF2 indel versions and IDS.IGF2delta30-40 resulted in a comparable therapeutic performance in all the regions, but not in cortex and corpus callosum, where IDS.IGF2 and IDS.IGF2 indel versions outperformed IDS.IGF2delta30-40.

Neuroinflammation is also characterized by activated microglia cells, which upregulate CD68. As a result, MPS II mice showed a widespread increase of CD68 deposition in the brain (FIG. 16). After lentiviral gene therapy, correction of altered CD68 deposition in MPS II mouse brain followed the same trend documented for Lamp1. IDS.IGF2, IDS.IGF2 indel versions and IDS.IGF2delta30-40 resulted in a comparable therapeutic performance, although IDS.IGF2 indel versions treatment appeared to result in better correction of CD68 deposition in cortex, hippocampus, hypothalamus and midbrain compared to IDS.IGF2delta30-40. VCN of the mice used for Alcian blue, Lamp1, GFAP and CD68 staining of the brain is shown in FIG. 18.

### REFERENCES

1. Langereis, E. J., Wagemans, T., Kulik, W., Lefeber, D. J., van Lenthe, H., Oussoren, E., van der Ploeg, A. T., Ruijter, G. J., Wevers, R. A., Wijburg, F. A., et al. (2015). A Multiplex Assay for the Diagnosis of Mucopolysaccharidoses and Mucolipidoses. PLOS One 10. 10.1371/JOURNAL.PONE.0138622.
2. Pike-Overzet, K., Baum, C., Bredius, R. G. M., Cavazzana, M., Driessen, G. J., Fibbe, W. E., Gaspar, H. B., Hoeben, R. C., Lagresle-Peyrou, C., Lankester, A., et al. (2014). Successful RAG1-SCID gene therapy depends on the level of RAG1 expression. Journal of Allergy and Clinical Immunology 134, 242-243. 10.1016/J.JACI.2014.04.033.
3. Bergsma, A. J., Stijn, •, in't Groen, L. M., Catalano, F., Yamanaka, M., Takahashi, S., Okumiya, T., Ans, •, van der Ploeg, T., and Pim Pijnappel, •W W M (2021). A generic assay for the identification of splicing variants that induce nonsense-mediated decay in Pompe disease. European Journal of Human Genetics 29, 422-433. 10.1038/s41431-020-00751-3.
4. Human Iduronate 2-Sulfatase/IDS DuoSet ELISA DY2449-05: R&D Systems https://www.rndsystems.com/products/human-iduronate-2-sulfatase-ids-duoset-elisa_dy2449-05.
5. Gleitz, H. F., Liao, A. Y., Cook, J. R., Rowlston, S. F., Forte, G. M., D'Souza, Z., O'Leary, C., Holley, R. J., and Bigger, B. W. (2018). Brain-targeted stem cell gene therapy corrects mucopolysaccharidosis type II via multiple mechanisms. EMBO Mol Med 10. 10.15252/emmm.201708730.
6. Alliegro, M., Ferla, R., Nusco, E., de Leonibus, C., Settembre, C., and Auricchio, A. (2016). Low-dose Gene Therapy Reduces the Frequency of Enzyme Replacement Therapy in a Mouse Model of Lysosomal Storage Disease. Mol Ther 24, 2054-2063. 10.1038/MT.2016.181.
7. Doyle, B. M., Turner, S. M. F., Sunshine, M. D., Doerfler, P. A., Poirier, A. E., Vaught, L. A., Jorgensen, M. L., Falk, D. J., Byrne, B. J., and Fuller, D. D. (2019). AAV Gene Therapy Utilizing Glycosylation-Independent Lysosomal Targeting Tagged GAA in the Hypoglossal Motor System of Pompe Mice. Mol Ther Methods Clin Dev 15, 194-203. 10.1016/J.OMTM.2019.08.009.
8. Voznyi, Y. v., Keulemans, J. L. M., and van Diggelen, O. P. (2001). A fluorimetric enzyme assay for the diagnosis of MPS II (hunter disease). J Inherit Metab Dis 24, 675-680. 10.1023/A: 1012763026526.

1. A nucleic acid molecule comprising:
a nucleotide sequence encoding a lysosomal hydrolase or an enzymatically active part thereof or a sequence having at least 90% sequence identity to said lysosomal hydrolase or part thereof,
a human insulin-like growth factor II (IGFII) gene sequence comprising a deletion of the nucleotides encoding amino acids 30-40, 30-41, 29-30 or 29-41 of human mature IGFII, and
a nucleotide sequence encoding at least one peptide that facilitates cellular uptake or transcytosis which is inserted at a location between the nucleotides encoding amino acids 28 and 42 of mature IGFII of said IGFII gene sequence at the location of the deletion of the IGFII gene sequence.

2. The nucleic acid molecule according to claim 1 wherein said lysosomal hydrolase is selected from the group consisting of human iduronate-2-sulfatase (IDS), acid alpha glucosidase (GAA), and tripeptidyl peptidase 1 (TPP1).

3. The nucleic acid molecule according to claim 1, wherein said lysosomal hydrolase:
is IDS and said nucleotide sequence encodes an amino acid sequence comprising a sequence having at least 90% identity with amino acids 26-550 of human IDS,
is GAA and said nucleotide sequence encodes an amino acid sequence comprising a sequence having at least 90% sequence identity with amino acids 70-952 of human GAA, or
is TPP1 and said nucleotide sequences encodes an amino acid sequence comprising a sequence having at least 90% sequence identity with amino acids 20-563 of human TPP1.

4. The nucleic acid molecule according to claim 1, wherein said peptide that facilitates cellular uptake or transcytosis is a peptide that facilitates passage over the blood brain barrier (BBB).

5. The nucleic acid molecule according to claim 4, wherein the peptide that facilitates passage over the BBB, comprises a low density lipoprotein receptor-related protein 1 (LRP1) binding domain and/or encodes a receptor binding domain of human apolipoprotein E2 (ApoE2), human apolipoprotein B (ApoB), human receptor associated protein (RAP), or human leptin, or encodes CRP peptide (CRTIGPSVC) or Angiopep-2 (TFFYGGSRGKRNNFKTEEY), or a combination and/or repeat of any of these domains and sequences.

6. The nucleic acid molecule according to claim 4, wherein said nucleotide sequence encodes amino acids 141-149 of human ApoE2 (LRKLRKRLL), amino acids 3371-3409 of human ApoB (SSVIDALQYKLEGTTRLTRKRGLKLATALSLSNKFVEGS), amino acids 251-262 of human RAP (EAKIEKHNHYQK), amino acids 61-90 of human leptin (YQQILTSMPSRNVIQISNDLENLRDLLHVL), or a combination and/or repeat of any of these sequences.

7. The nucleic acid molecule according to claim 1, wherein said human IGFII gene sequence comprises a nucleotide sequence encoding amino acids 8-28 and 42-67 of human mature IGFII, amino acids 8-29 and 41-67 of human mature IGFII, preferably amino acids 1, 8-28 and 42-67 of human mature IGFII, or amino acids 1, 8-29 and 41-67 of human mature IGFII, or sequences having at least 90% sequence identity thereto.

8. The nucleic acid molecule according to claim 1, comprising nucleotides encoding at least a cysteine at each side of the nucleotide sequence encoding at least one peptide that facilitates cellular uptake or transcytosis and/or comprising a linking sequence at one or both sides of the nucleotide sequence encoding at least one peptide that facilitates cellular uptake or transcytosis.

9. (canceled)

10. The nucleic acid molecule according to claim 1, comprising a linking sequence and nucleotides encoding a cysteine at both sides of the nucleotide sequence encoding at least one peptide that facilitates cellular uptake or transcytosis, whereby the linking sequence is adjacent to the nucleotide sequence encoding at least one peptide that facilitates cellular uptake or transcytosis.

11. The nucleic acid molecule according to claim 1, which is a gene therapy vector.

12. A viral particle comprising a nucleic acid molecule according to claim 1.

13. A fusion protein encoded by the nucleic acid molecule according to claim 1.

14. A cell population provided with a nucleic acid molecule of claim 1 or with a viral particle comprising said nucleic acid molecule.

15. (canceled)

16. A method of treatment of a lysosomal storage disorder comprising administering the nucleic acid molecule according to claim 1, a viral particle comprising the nucleic acid molecule or a cell population provided with the nucleic acid molecule to an individual in need thereof.

17. The method according to claim 16 comprising removing cells from the individual, providing said cells with the nucleic acid molecule according to claim 1 or a viral particle comprising the nucleic acid molecule, and administering said cells provided with said nucleic acid molecule or viral particle to said individual.

18. The fusion protein according to claim 13, wherein the fusion protein comprises a lysosomal hydrolase or an enzymatically active part thereof, or a sequence having at least 90% sequence identity to said lysosomal hydrolase or part thereof, fused to a human insulin-like growth factor II (IGFII) sequence comprising a deletion of the nucleotides encoding amino acids 30-40 or 29-41 of human mature IGFII and at least one peptide that facilitates cellular uptake or transcytosis, wherein said lysosomal hydrolase or part thereof or sequence having at least 90% sequence identity to said lysosomal hydrolase or part thereof, said human IGFII sequence and/or said at least one peptide that facilitates cellular uptake or transcytosis are separated by one or more linking sequences.

19. The cell population according to claim 14, wherein the cell population comprises hematopoietic stem cells (HSCs).

20. The cell population according to claim 14, wherein the cells are transfected or transduced with said nucleic acid molecule or viral particle.

21. The cell population according to claim 20 wherein the cells express a fusion protein comprising a lysosomal hydrolase or an enzymatically active part thereof or a sequence having at least 90% sequence identity to said lysosomal hydrolase or part thereof fused to a human insulin-like growth factor II (IGFII) sequence and at least one peptide that facilitates cellular uptake or transcytosis.

22. A method of treatment of a lysosomal storage disorder comprising administering the fusion protein according to claim 13 to an individual in need thereof.
